(19) United States
(12) Patent Application Publication
(10) Pub. No.: US 2025/0275542 A1
(43) Pub. Date: Sep. 4, 2025

(54) Baked and Par-Baked Products with Thermostable AMG Variants from Penicillium

(71) Applicant: Novozymes A/S, Bagsvaerd (DK)

(72) Inventors: Henrik Lundkvist, Malmo (SE); Camila Varming, Broenshoej (DK); Carsten Andersen, Vaerloese (DK); Hasim Sinik, Istanbul (TR); Esra Ozcomlekci, Istanbul (TR)

(73) Assignee: NOVOZYMES A/S, Bagsvaerd (DK)

(21) Appl. No.: 18/250,397

(22) PCT Filed: Nov. 2, 2021
(86) PCT No.: PCT/EP2021/080317

§ 371 Date: Apr. 25, 2023

(30) Foreign Application Priority Data
Nov. 2, 2020 (DK) PA 2020 01238
Apr. 12, 2021 (DK) PA 2021 00367

(51) Int. Cl. A21D 8/04; C12N 9/34

(52) U.S. Cl. CPC A21D 8/042; C12N 9/2428; C12Y 302/01003

(57) ABSTRACT

The invention relates to methods of producing a baked or par-baked product, said method comprising a first step of providing a dough comprising a mature thermostable variant of a parent glucoamylase at least 70% identical to SEQ ID NO:1, SEQ ID NO:6, SEQ ID NO:7 or SEQ ID NO:8; and a second step of baking or par-baking the dough to produce a baked or par-baked product, as well as baking compositions comprising said variant and uses of said variant.

### CROSS-REFERENCE TO RELATED APPLICATIONS

This application is a 35 U.S.C. 371 national application of international application no. PCT/EP2021/080317 filed Nov. 2, 2021, which claims priority or the benefit under 35 U.S.C. 119 of Danish application nos. PA 2020 01238 and PA 2021 00367 filed Nov. 2, 2020 and Apr. 12, 2021, the contents of which are fully incorporated herein by reference.

### REFERENCE TO SEQUENCE LISTING

This application contains a Sequence Listing in computer readable form, which is incorporated herein by reference.

### FIELD OF THE INVENTION

The invention relates to methods of producing a baked or par-baked product, said method comprising a first step of providing a dough comprising a mature thermostable variant of a parent glucoamylase at least 70% identical to SEQ ID NO:1, SEQ ID NO:6, SEQ ID NO:7 or SEQ ID NO:8; and a second step of baking or par-baking the dough to produce a baked or par-baked product, as well as baking compositions comprising said variant and uses of said variant.

### BACKGROUND OF THE INVENTION

World-wide, baked products (breads, biscuits, etc.) containing sugar is one of the most popular product segments. The recipe amount of sugar will typically be 1-25% of total flour weight.

However, due to increased market price for sugar, shortage in sugar availability in some parts of the world as well as health-concerns, there is a need for methods of producing baked products that contain a reduced amount of added sugar without sacrificing the quality of the baked product and perhaps even improving it.

WO 2019/238423 (Novozymes A/S, Denmark) discloses methods of producing a dough with a reduced amount of added sugar comprising adding a raw starch degrading alpha-amylase and a glucoamylase to the dough ingredients.

### SUMMARY OF THE INVENTION

The inventors found that thermostabilized variants of certain glucoamylases showed greatly improved performance in freshkeeping or anti-staling of a baked or par-baked product. Another improved performance of the thermostabilized variants was that they increased the sweetness or sweet taste of the product, which allowed a reduction in the amount of added sugar in traditional recipes.

Accordingly in a first aspect, the invention relates to method of producing a baked or par-baked product, said method comprising:
a) providing a dough comprising a mature thermostable variant of a parent glucoamylase at least 70% identical to SEQ ID NO:1, SEQ ID NO:6, SEQ ID NO:7 or SEQ ID NO:8; and
b) baking or par-baking the dough to produce a baked or par-baked product.

A second aspect of the invention, relates to baking compositions comprising a mature thermostable variant of a parent glucoamylase as defined in the first aspect.

Other aspects of the invention relate to uses of the baking compositions of the second aspect for sugar replacement in a method of producing a baked or par-baked product, for increasing the sweetness of a baked or par-baked product, for reducing the amount of sugar in the dough in a method of producing a baked or par-baked product and/or for extending the shelf-life of a baked or par-baked product in a method of producing a baked or par-baked product, as well as in methods as defined in the first aspect, whereby the baked or par-baked product after final bake-off has a reduced initial firmness and/or an increased initial elasticity, and/or a reduced increase in firmness and/or a higher elasticity after 1, 7 or 14 days, when cooled to room temperature, packed in a sealed container and stored at room temperature until analysis, compared to a control made without any added glucoamylase.

Preferably, the mature thermostable variant of a parent glucoamylase of the invention is at least 71% identical to SEQ ID NO:1, SEQ ID NO:6, SEQ ID NO:7 or SEQ ID NO:8, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identical to SEQ ID NO:1, SEQ ID NO:6, SEQ ID NO:7 or SEQ ID NO:8.

### FIGURES

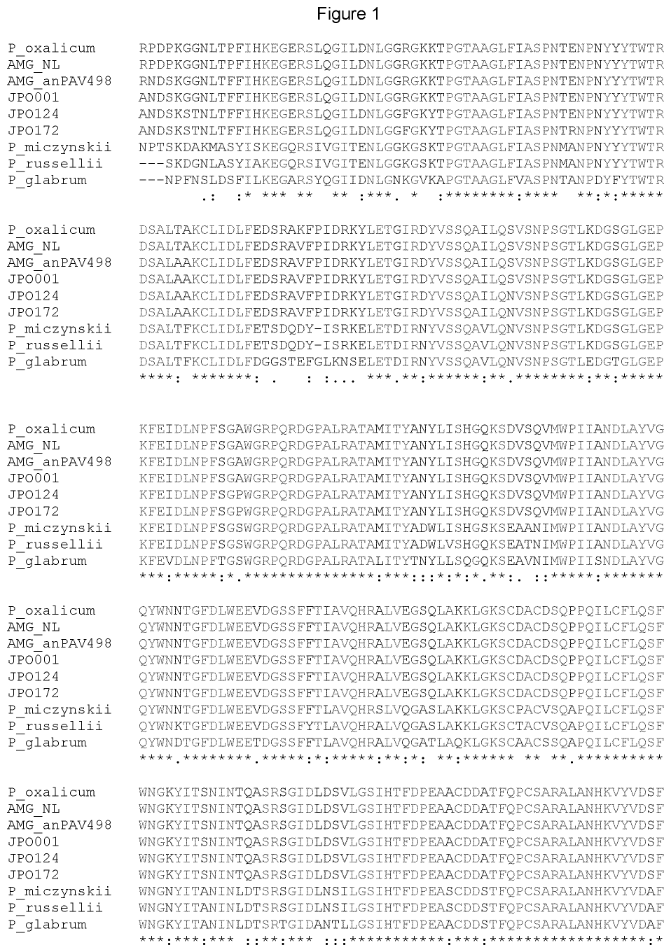

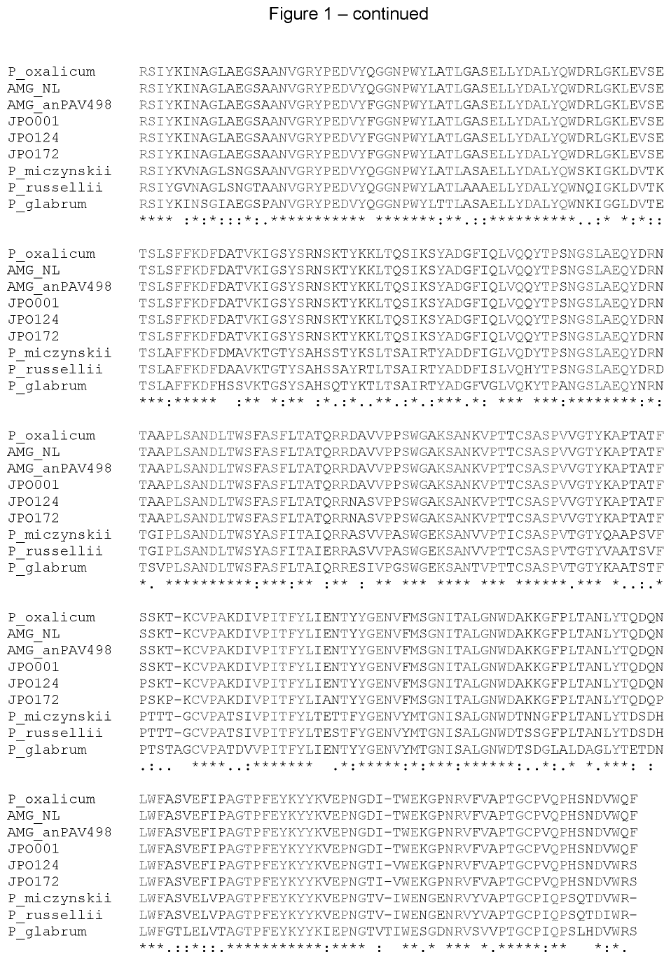

FIG. 1 shows a multiple alignment of the amino acid sequences of the mature proteins of:
Wildtype AMG from Penicillium oxalicum (PoAMG) of SEQ ID NO:1
PoAMG variant denoted ‘AMG NL’ of SEQ ID NO:2
PoAMG variant denoted ‘AMG anPAV498’ of SEQ ID NO:3
PoAMG variant denoted ‘AMG JPO001’ of SEQ ID NO:4
PoAMG variant denoted ‘AMG JPO124’ of SEQ ID NO:5
PoAMG variant denoted ‘AMG JPO172’ of SEQ ID NO:6
Wildtype AMG from Penicillium miczynskii (PoAMG) of SEQ ID NO:7
Wildtype AMG from Penicillium russellii (PoAMG) of SEQ ID NO:8
Wildtype AMG from Penicillium glabrum (PoAMG) of SEQ ID NO:9

### DETAILED DESCRIPTION OF THE INVENTION

### Definitions

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labelled “longest identity” (obtained using the—no brief option) is used as the percent identity and is calculated as follows:

(Identical Residues×100)/(Length of Alignment−Total Number of Gaps in Alignment)

Variant: The term “variant” means a polypeptide comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more amino acids adjacent to and immediately following the amino acid occupying a position. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope, or a binding domain. Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Increased strength: The term “increased strength of the dough” is defined herein as the property of a dough that has generally more elastic properties and/or requires more work input to mould and shape compared to a control.

Increased elasticity: The term “increased elasticity of the dough” is defined herein as the property of a dough which has a higher tendency to regain its original shape after being subjected to a certain physical strain compared to a control.

Increased stability of the dough: The term “increased stability of the dough” is defined herein as the property of a dough that is less susceptible to mechanical abuse thus better maintaining its shape and volume and is evaluated by the ratio of height:width of a cross section of a loaf after normal and/or extended proof compared to a control.

Reduced stickiness of the dough: The term “reduced stickiness of the dough” is defined herein as the property of a dough that has less tendency to adhere to surfaces compared to a control, e.g., in the dough production machinery, and it is either evaluated empirically by the skilled test baker or measured by, e.g., a texture analyser (e.g. TAXT2) as known in the art.

Improved extensibility: The term “improved extensibility of the dough” is defined herein as the property of a dough that can be subjected to increased strain or stretching without rupture compared to a control.

Improved machinability: The term “improved machinability of the dough” is defined herein as the property of a dough that is generally less sticky and/or firmer and/or more elastic compared to a control.

Increased volume of the baked product: The term “increased volume of the baked product” is measured as the volume of a given loaf of bread compared to a control. The volume may be determined as known in the art.

Improved crumb structure of the baked product: The term “improved crumb structure of the baked product” is defined herein as the property of a baked product with finer cells and/or thinner cell walls in the crumb and/or more uniform/homogenous distribution of cells in the crumb compared to a control and is usually evaluated visually by the skilled baker or by digital image analysis as known in the art (e. g., C-cell, Calibre Control International Ltd, Appleton, Warrington, UK).

Improved softness of the baked product: The term “improved softness of the baked product” is the opposite of “firmness” and is defined herein as the property of a baked product that is more easily compressed compared to a control and is evaluated either empirically by the skilled test baker or measured by, e.g., a texture analyser (e.g. TAXT2 or TA-XT Plus from Stable Micro Systems Ltd, surrey, UK) as known in the art.

Sensory attributes of the baked products: The sensory attributes may be evaluated using procedures well established in the baking industry, and may include, for example, the use of a panel of trained taste-testers.

Thermostability improvement: The thermostability improvement (Td) in ° C. is a measure of how much the variants have improved in thermostability over their parent glucoamylase under the same conditions, determined as exemplified herein.

The first aspect of the invention relates to method of producing a baked or par-baked product, said method comprising:
a) providing a dough comprising a mature thermostable variant of a parent glucoamylase at least 70% identical to SEQ ID NO:1, SEQ ID NO:6, SEQ ID NO:7 or SEQ ID NO:8; and
b) baking or par-baking the dough to produce a baked or par-baked product.

A second aspect of the invention, relates to baking compositions comprising a mature thermostable variant of a parent glucoamylase as defined in the first aspect.

Other aspects of the invention relate to uses of the baking compositions of the second aspect for sugar replacement in a method of producing a baked or par-baked product, for increasing the sweetness of a baked or par-baked product, for reducing the amount of sugar in the dough in a method of producing a baked or par-baked product and/or for extending the shelf-life of a baked or par-baked product in a method of producing a baked or par-baked product, as well as in methods as defined in the first aspect, whereby the baked or par-baked product after final bake-off has a reduced initial firmness and/or an increased initial elasticity, and/or a reduced increase in firmness and/or a higher elasticity after 1, 7 or 14 days, when cooled to room temperature, packed in a sealed container and stored at room temperature until analysis, compared to a control made without any added glucoamylase.

Preferably, the mature thermostable variant of a parent glucoamylase of the invention is at least 71% identical to SEQ ID NO:1, SEQ ID NO:6, SEQ ID NO:7 or SEQ ID NO:8, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identical to SEQ ID NO:1, SEQ ID NO:6, SEQ ID NO:7 or SEQ ID NO:8.

### The Dough

As used herein “dough” means any dough used to prepare a baked product, in particular a bread.

According to the present invention, the dough used to prepare a baked product may be made from any suitable dough ingredients comprising flour.

The flour may be from any baking grain known in the art, such as, wheat flour, corn flour, rye flour, barley flour, oat flour, rice flour, sorghum flour, potato flour, soy flour, and any combinations thereof (e.g., wheat flour combined with one of the other flour sources; or rice flour combined with one of the other flour sources).

In a preferred embodiment, the flour is wheat flour.

In a preferred embodiment, at least 10% (w/w) or more of the total flour content is wheat flour, e.g., at least 15% or more of the total flour content is wheat flour, e.g., at least 20% or more of the total flour content is wheat flour, e.g., at least 25% or more of the total flour content is wheat flour, e.g., at least 30% or more of the total flour content is wheat flour, e.g., at least 35% or more of the total flour content is wheat flour, e.g., at least 40% or more of the total flour content is wheat flour, e.g., at least 45% or more of the total flour content is wheat flour, e.g., at least 50% or more of the total flour content is wheat flour, e.g., at least 55% or more of the total flour content is wheat flour, e.g., at least 60% or more of the total flour content is wheat flour, e.g., at least 65% or more of the total flour content is wheat flour, e.g., at least 70% or more of the total flour content is wheat flour, e.g., at least 75% or more of the total flour content is wheat flour, e.g., at least 80% or more of the total flour content is wheat flour, e.g., at least 85% or more of the total flour content is wheat flour, e.g., at least 90% or more of the total flour content is wheat flour, e.g., at least 95% or more of the total flour content is wheat flour, e.g., 100% of total the flour is wheat flour.

The dough of the invention is normally a leavened dough or a dough to be subjected to leavening. The dough may be leavened in various ways, such as by adding dough ingredients such as chemical leavening agents, e.g., sodium bicarbonate or by adding a leaven (fermenting dough), but it is preferred to leaven the dough by adding a suitable yeast culture, such as a culture of Saccharomyces cerevisiae (baker's yeast), e.g., a commercially available strain of S. cerevisiae.

The dough of the invention may typically comprise some added sugar as the method according to the invention is able to reduce the amount of added sugar, but normally a partially reduction of sugar is obtained.

In one embodiment, the amount of added sugar is reduced by at least 10% (w/w) compared to the amount of sugar added to a dough in an original recipe, e.g., the amount of added sugar is reduced by at least 20% (w/w) compared to the amount of sugar added to a dough in an original recipe, e.g., the amount of added sugar is reduced by at least 30% (w/w) compared to the amount of sugar added to a dough in an original recipe, e.g., the amount of added sugar is reduced by at least 40% (w/w) compared to the amount of sugar added to a dough in an original recipe, e.g., the amount of added sugar is reduced by at least 50% (w/w) compared to the amount of sugar added to a dough in an original recipe, e.g., the amount of added sugar is reduced by at least 60% (w/w) compared to the amount of sugar added to a dough in an original recipe, e.g., the amount of added sugar is reduced by at least 70% (w/w) compared to the amount of sugar added to a dough in an original recipe, e.g., the amount of added sugar is reduced by at least 80% (w/w) compared to the amount of sugar added to a dough in an original recipe, e.g., the amount of added sugar is reduced by at least 90% (w/w) compared to the amount of sugar added to a dough in an original recipe, e.g., the amount of added sugar is reduced by 100% (w/w) compared to the amount of sugar added to a dough in an original recipe.

The dough may also comprise other conventional dough ingredients, e.g., proteins, such as milk powder, gluten, and soy; eggs (either whole eggs, egg yolks or egg whites); an oxidant such as ascorbic acid, potassium bromate, potassium iodate, azodicarbonamide (ADA) or ammonium persulfate; an amino acid such as L-cysteine; a salt such as sodium chloride, calcium acetate, sodium sulphate, calcium sulphate, diluents such as silica dioxide, and starch of different origins. Still other conventional ingredients include hydrocolloids such as CMC, guar gum, xanthan gum, locust bean gum, etc.

The dough ingredients may typically comprise fat (triglyceride) and/or oil and/or shortenings, in particular oil such as sunflower oil or rapeseed oil.

The dough may be prepared applying any conventional mixing process, such as the continuous mix process, straight-dough process, or the sponge and dough method.

The present invention is particularly useful for preparing dough and baked products in industrialized processes in which the dough used to prepare the baked products are prepared mechanically using automated or semi-automated equipment.

The process of preparing bread generally involves the sequential steps of dough making, sheeting or dividing, shaping or rolling, and proofing the dough, which steps are well known in the art.

As used herein, “baked product” means any kind of baked product including bread types such as pan bread, toast bread, open bread, pan bread with and without lid, buns, Fino bread, Hammam bread, Samoli bread, baguettes, brioche hamburger buns, rolls, brown bread, whole meal bread, rich bread, bran bread, flat bread, tortilla, biscuits, and any variety thereof. According to the present invention, the baked product may also be a cake or any patisserie product as known in the art.

### Raw Starch Degrading Alpha-Amylase

As used herein, a “raw starch degrading alpha-amylase” refers to an enzyme that can directly degrade raw starch granules below the gelatinization temperature of starch.

Examples of raw starch degrading alpha-amylases include the ones disclosed in WO 2005/003311, U.S. Patent Publication no. 2005/0054071, and U.S. Pat. No. 7,326,548. Examples also include those enzymes disclosed in Table 1 to 5 of the examples in U.S. Pat. No. 7,326,548, in U.S. Patent Publication no. 2005/0054071 (Table 3 on page 15), as well as the enzymes disclosed in WO 2004/020499 and WO 2006/06929 and WO 2006/066579.

In one embodiment, the raw starch degrading alpha-amylase is a GH13_1 amylase.

In one embodiment, the raw starch degrading alpha-amylase enzyme has at least 70%, e.g. at least 71%, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identity to the raw starch degrading alpha-amylase shown in EP U.S. Pat. No. 2,981,170 (Novozymes A/S).

In one embodiment, the raw starch degrading alpha-amylase according to the invention may be added to flour or dough in an amount of 0.01-10 mg enzyme protein per kg flour, e.g., in an amount of 0.1-5 mg enzyme protein per kg flour.

### Glucoamylases

Glucoamylases are also called amyloglucosidases, and Glucan 1,4-alpha-glucosidase (EC 3.2.1.3), more commonly they are referred to as AMGs.

According to the present invention, different types of amyloglucosidases may be used as parent for the generation of a thermostable amyloglucosidase variant, e.g, the amyloglucosidase may be a polypeptide that is encoded by a DNA sequence that is found in a fungal strain of Aspergillus, Rhizopusor, Talaromyces or Penicillium; preferably the DNA sequence that is found in a fungal strain of Penicillium, even more preferably the DNA sequence that is found in a fungal strain of Penicillium oxysporum, Penicillium oxalicum, Penicillium miczynskii, Penicillium russellii or Penicillium glabrum. Preferably, the parent glucoamylase is from a species of Penicillium, preferably from Penicillium oxicalum, Penicillium miczynskii, Penicillium russellii or Penicillium glabrum.

Examples of other suitable fungi include Aspergillus niger, Aspergillus awamori, Aspergillus oryzae, Rhizopus delemar, Rhizopus niveus, Rhizopus oryzae and Talaromyces emersonii.

Below is shown the %-identity between the AMG amino acid sequences aligned in FIG. 1, and also provided in the sequence list:

| | | | | | | | | | |
|---|---|---|---|---|---|---|---|---|---|
| P_{—}oxalicum | 100.00 | 99.83 | 98.99 | 98.82 | 96.64 | 95.97 | 77.07 | 77.12 | 74.32 |
| AMG_NL | 99.83 | 100.00 | 99.16 | 98.99 | 96.81 | 96.13 | 77.07 | 77.12 | 74.32 |
| AMG_anPAV498 | 98.99 | 99.16 | 100.00 | 99.83 | 97.65 | 96.97 | 76.73 | 76.95 | 73.82 |
| AMG_JPO001 | 98.82 | 98.99 | 99.83 | 100.00 | 97.82 | 97.14 | 76.73 | 76.95 | 73.82 |
| AMG_JPO124 | 96.64 | 96.81 | 97.65 | 97.82 | 100.00 | 99.33 | 77.07 | 77.12 | 74.32 |
| AMG_JPO172 | 95.97 | 96.13 | 96.97 | 97.14 | 99.33 | 100.00 | 76.73 | 76.78 | 73.99 |
| P_{—}miczynskii | 77.07 | 77.07 | 76.73 | 76.73 | 77.07 | 76.73 | 100.00 | 94.75 | 80.51 |
| P_{—}russellii | 77.12 | 77.12 | 76.95 | 76.95 | 77.12 | 76.78 | 94.75 | 100.00 | 79.66 |
| P_{—}glabrum | 74.32 | 74.32 | 73.82 | 73.82 | 74.32 | 73.99 | 80.51 | 79.66 | 100.00 |

In one embodiment, the glucoamylase according to the invention may be added to flour or dough in an amount 0.01-1,000 mg enzyme protein (mgEP) per kg flour, preferably in an amount of 0.01-500 mg enzyme protein (mgEP) per kg flour, even more preferably in an amount of 0.1-100 mg enzyme protein (mgEP) per kg flour.

Thermostable variants of the PoAMG have been generated (see table 2 below). In a preferred embodiment, the mature thermostable glucoamylase variant of the invention comprises one or more or all of the combinations of amino acid substitutions listed in table 2 below.

In a preferred embodiment, the mature variant of the invention comprises at least one amino acid modification in one or more or all of the positions corresponding to positions 1, 2, 4, 6, 7, 11, 31, 34, 65, 79, 103, 132, 327, 445, 447, 481, 566, 568, 594 and 595 in SEQ ID NO:1; preferably the at least one amino acid modification comprises a substitution in one or more or all of the positions corresponding to positions 1, 2, 4, 11, 65, 79 and 327 in SEQ ID NO:1, preferably the at least one amino acid modification comprises a substitution in one or more or all of the positions corresponding to R1A, P2N, P4S, P11F, T65A, K79V and Q327F in SEQ ID NO:1; or preferably the at least one amino acid modification comprises a substitution in one or more or all of the positions corresponding to positions 1, 6, 7, 31, 34, 79, 103, 132, 445, 447, 481, 566, 568, 594 and 595 in SEQ ID NO:1, preferably the at least one amino acid modification comprises a substitution in one or more or all of the positions corresponding to R1A, G6S, G7T, R31F, K34Y, K79V, S103N, A132P, D445N, V447S, S481P, D566T, T568V, Q594R and F595S in SEQ ID NO:1; or preferably the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1, preferably the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1.

The thermostability improvements (Td) of the variants in table 2 are listed in Table 3, where the Td of the PoAMG variant denoted “anPAV498” (the parent) was set to zero. In a preferred embodiment, the mature thermostable variant of the invention has a thermostability improvement (Td) over its parent of at least 3° C., preferably at least 4° C., 5° C., 6° C., 7° C. or 8° C., preferably determined as exemplified herein.

In another preferred embodiment, the mature thermostable variant of the invention has a relative activity at 91° C. of at least 150, preferably at least 200, more preferably at least 250, most preferably at least 300 compared to its parent.

Preferably, the mature thermostable variant glucoamylase enzyme is comprised in the dough in an amount of 0.01-1,000 mg enzyme protein (mgEP) per kg flour, preferably in an amount of 0.01-500 mg enzyme protein (mgEP) per kg flour, even more preferably in an amount of 0.1-100 mg enzyme protein (mgEP) per kg flour.

### Amylases

Alpha-Amylases (alpha-1,4-glucan-4-glucanohydrolases, EC. 3.2.1.1) constitute a group of enzymes which catalyze hydrolysis of starch and other linear and branched 1,4-glucosidic oligo- and polysaccharides.

A number of alpha-amylases are referred to as Termamyl™ and “Termamyl™-like alpha-amylases” and are known from, e.g., WO 90/11352, WO 95/10603, WO 95/26397, WO 96/23873 and WO 96/23874.

Another group of alpha-amylases are referred to as Fungamyl™ and “Fungamyl™-like alpha-amylases”, which are alpha-amylases related to the alpha-amylase derived from Aspergillus oryzae disclosed in WO 01/34784.

Suitable commercial alpha-amylase compositions according to the present invention include, e.g., BAKEZYME P 300 (available from DSM) and FUNGAMYL 2500 SG, FUNGAMYL 4000 BG, FUNGAMYL 4000 SG, FUNGAMYL 800 L, FUNGAMYL ULTRA BG and FUNGAMYL ULTRA SG (available from Novozymes A/S).

In one embodiment, the alpha-amylase according to the invention may be added to flour or dough in an amount of 0.01-1,000 mg enzyme protein (mgEP) per kg flour, preferably in an amount of 0.01-500 mg enzyme protein (mgEP) per kg flour, even more preferably in an amount of 0.1-100 mg enzyme protein (mgEP) per kg flour.

### Additional Enzymes

Optionally, one or more additional enzymes, such as alpha-amylase, maltogenic amylase, beta amylase, aminopeptidase, carboxypeptidase, catalase, cellulytic enzyme, chitinase, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase may be used together with the enzyme composition according to the invention.

The additional enzyme(s) may be of any origin, including mammalian, plant, and microbial (bacterial, yeast or fungal) origin.

The maltogenic alpha-amylase (EC 3.2.1.133) may be from Bacillus. A maltogenic alpha-amylase from B. stearothermophilus strain NCIB 11837 is commercially available from Novozymes A/S under the tradename Novamyl®.

The maltogenic alpha-amylase may also be a variant of the maltogenic alpha-amylase from B. stearothermophilus as disclosed in, e.g., WO1999/043794; WO2006/032281; or WO2008/148845, e.g., Novamyl® 3D.

An anti-staling amylase for use in the invention may also be an amylase (glucan 1,4-alpha-maltotetrahydrolase (EC 3.2.1.60)) from Pseudomonas saccharophilia or variants thereof, such as any of the amylases disclosed in WO1999/050399, WO2004/111217 or WO2005/003339.

The glucose oxidase may be a fungal glucose oxidase, in particular an Aspergillus niger glucose oxidase (such as GLUZYME®, available from Novozymes A/S).

The xylanase which may be of microbial origin, e.g., derived from a bacterium or fungus, such as a strain of Aspergillus, in particular of A. aculeatus, A. niger, A. awamori, or A. tubigensis, from a strain of Trichoderma, e.g. T. reesei, or from a strain of Humicola, e.g., H. insolens.

Suitable commercially available xylanase preparations for use in the present invention include PANZEA BG, PENTOPAN MONO BG and PENTOPAN 500 BG (available from Novozymes A/S), GRINDAMYL POWERBAKE (available from Danisco), and BAKEZYME BXP 5000 and BAKEZYME BXP 5001 (available from DSM).

The protease may be from Bacillus, e.g., B. amyloliquefaciens. A suitable protease may be Neutrase® available from Novozymes A/S.

The phospholipase may have phospholipase A1, A2, B, C, D or lysophospholipase activity; it may or may not have lipase activity. It may be of animal origin, e.g. from pancreas, snake venom or bee venom, or it may be of microbial origin, e.g., from filamentous fungi, yeast or bacteria, such as Aspergillus or Fusarium, e.g., A. niger, A. oryzae or F. oxysporum. A preferred lipase/phospholipase from Fusarium oxysporum is disclosed in WO 98/26057. Also, the variants described in WO 00/32758 may be used.

Suitable phospholipase compositions are LIPOPAN F, LIPOPAN XTRA, and LIPOPAN MAX (available from Novozymes A/S) or PANAMORE GOLDEN and PANAMORE SPRING (available from DSM).

Preferably, the one or more additional enzyme is added in an amount of 0.01-1,000 mg enzyme protein (mgEP) per kg flour, preferably in an amount of 0.01-500 mg enzyme protein (mgEP) per kg flour, even more preferably in an amount of 0.1-100 mg enzyme protein (mgEP) per kg flour.

### Enzyme Compositions

The mature thermostable variant glucoamylase of the invention as well as any additional enzyme(s) may be added to flour or dough in any suitable form, such as, e.g., in the form of a liquid, in particular a stabilized liquid, or it may be added to flour or dough as a substantially dry powder or granulate.

Granulates may be produced, e.g., as disclosed in U.S. Pat. Nos. 4,106,991 and 4,661,452. Liquid enzyme preparations may, for instance, be stabilized by adding a sugar or sugar alcohol or lactic acid according to established procedures. Other enzyme stabilizers are well-known in the art.

The enzyme(s) may be added to the bread dough ingredients in any suitable manner, such as individual components (separate or sequential addition of the enzymes) or addition of the enzymes together in one step or one composition.

### Baking Composition

The present invention further relates to baking compositions comprising a mature thermostable variant of a parent glucoamylase as defined in the first aspect of the invention.

The baking composition may contain other dough-improving and/or bread-improving additives, e.g., any of the additives, including enzymes, mentioned above.

The baking composition may be, e.g., a dough composition, a flour composition, a flour pre-mix, or a bread improver.

Preferably, the baking compositions of the invention also comprise one or more additional enzyme selected from the group consisting of a alpha-amylase, maltogenic amylase, beta amylase, aminopeptidase, carboxypeptidase, catalase, cellulytic enzyme, chitinase, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase.

Preferably, the baking compositions of the invention also comprise flour, sugar, yeast, salt and/or fat.

It will often be advantageous to provide the enzymes used in the treatment of the present invention in admixture with other ingredients used to improve the properties of baked products. These baking compositions are commonly known in the art as “pre-mixes,” which usually comprise flour.

Hence, in a further aspect, the present invention relates to a bread premix for improving the quality of dough by reducing the amount of added sugar, which premix comprises the enzyme combination of the present invention.

In one embodiment, the present invention further relates to a bread pre-mix comprising the enzyme combination of the present invention and flour, such as, flour from grains, such as, wheat flour, corn flour, rye flour, barley flour, oat flour, rice flour, or sorghum flour, and combinations thereof.

In another embodiment, the present invention relates to a bread pre-mix comprising the enzyme combination of the present invention and flour, such as, flour from grains, such as, wheat flour, corn flour, rye flour, barley flour, oat flour, rice flour, sorghum, soy flour, and combinations thereof, and one or more additional enzymes, as previously described.

The pre-mix may be in the form of a granulate or agglomerated powder, e.g., wherein typically 95% (by weight) of the granulate or agglomerated powder has a particle size between 25 and 500 μm.

Granulates and agglomerated powders may be prepared by conventional methods, e.g., by spraying the enzymes onto a carrier in a fluid-bed granulator. The carrier may consist of particulate cores having a suitable particle size. The carrier may be soluble or insoluble, e.g. a salt (such as NaCl or sodium sulfate), a sugar (such as sucrose or lactose), a sugar alcohol (such as sorbitol), starch, rice, corn grits, or soy.

### Bread Properties

Organoleptic qualities or sensory attributes of the bread may be measured as known in the art. The properties of the bread may be referred to herein as sensory attributes, which include anti-staling (bread crumb firmness/hardness), crumb properties and mouth feel, or more precisely, the attributes of bread as detected in the mouth during eating (e.g., bread softness/resistance to first bite, crumb moistness, crumb chewiness and gumminess, and crumb smoothness and melting properties).

In one embodiment, the sensory attribute of the baked product is an increased sweetness by using the enzyme solution according to the invention.

In one embodiment, the sensory attribute of the baked product is an increased crumb sweetness by using the enzyme solution according to the invention.

In a preferred embodiment of the invention, the baked or par-baked product after final bake-off has a reduced initial firmness and/or an increased initial elasticity, and/or a reduced increase in firmness and/or a higher elasticity after 1, 7 or 14 days, when cooled to room temperature, packed in a sealed container and stored at room temperature until analysis, compared to a control made without any added glucoamylase.

In another preferred embodiment, the baked or par-baked product after final bake-off has at least the same sweetness or sweet taste as a control product made with double the amount of the mature glucoamylase the amino acid sequence of which is shown in SEQ ID NO:10, preferably determined as exemplified herein; preferably the baked or par-baked product after final bake-off has a higher sweetness or more sweet taste than a control product made with double the amount of the mature glucoamylase the amino acid sequence of which is shown in SEQ ID NO:10, preferably determined as exemplified herein.

The invention described and claimed herein is not to be limited in scope by the specific embodiments herein disclosed, since these embodiments are intended as illustrations of several aspects of the invention. Any equivalent embodiments are intended to be within the scope of this invention as well as combinations of one or more of the embodiments.

Various references are cited herein, the disclosures of which are incorporated by reference in their entireties. The present invention is further described by the following example which should not be construed as limiting the scope of the invention.

### EXAMPLES

### Example 1: Construction of PoAMG Libraries

PoAMG Libraries were Constructed as Follows:

A forward or reverse primer having NNK or desired mutation(s) at target site(s) with 15 bp overlaps each other were designed. Inverse PCR, which means amplification of entire plasmid DNA sequences by inversely directed primers, were carried out with appropriate template plasmid DNA (e.g. plasmid DNA containing JPO-0001 gene) by the following conditions. The resultant PCR fragments were purified by QIAquick Gel extraction kit [QIAGEN], and then introduced into Escherichia coli ECOS Competent E. coli DH5α [NIPPON GENE CO., LTD.]. The plasmid DNAs were extracted from E. coli transformants by MagExtractor plasmid extraction kit [TOYOBO], and then introduced into A. niger competent cells.

### PCR Reaction Mix:

PrimeSTAR Max DNA polymerase [TaKaRa]
Total 25 μl
1.0 μl Template DNA (1 ng/μl)
9.5 μl H₂O
12.5 μl 2× PrimeSTAR Max pre-mix
1.0 μl Forward primer (5 μM)
1.0 μl Reverse primer (5 μM)

### PCR Program:

98° C./2 min
25× (98° C./10 sec, 60° C./15 sec, 72° C./2 min)
10° C./hold

### Example 2: Screening for Better Thermostability

B. subtilis libraries constructed as in EXAMPLE 1 were fermented in either 96-well or 24-well MTP containing COVE liquid medium (2.0 g/L sucrose, 2.0 g/L iso-maltose, 2.0 g/L maltose, 4.9 mg/L, 0.2 ml/L 5N NaOH, 10 ml/L COVE salt, 10 ml/L 1M acetamide), 32° C. for 3 days. Then, AMG activities in culture supernatants were measured at several temperatures by pNPG assay described as follows.

pNPG Thermostability Assay:

The culture supernatants containing desired enzymes was mixed with same volume of pH 5.0 200 mM NaOAc buffer. Twenty microliter of this mixture was dispensed into either 96-well plate or 8-strip PCR tube, and then heated by thermal cycler at various temperatures for 30 min. Those samples were mixed with 10 μl of substrate solution containing 0.1% (w/v) pNPG [wako] in pH 5.0 200 mM NaOAc buffer and incubated at 70° C. for 20 min for enzymatic reaction. After the reaction, 60 μl of 0.1M Borax buffer was added to stop the reaction. Eighty microliter of reaction supernatant was taken out and its OD₄₀₅ value was read by photometer to evaluate the enzyme activity.

TABLE 1a
Lists of the relative activity of PoAMG variants
when compared with their parent anPAV498 or
JPO-0001 (anPAV498 w. leader-/propeptide)

| Name | Relative activity of 80° C./75° C. (%) |
|---|---|
| anPAV498 | 17% |
| JPO-004 | 32% |
| JPO-005 | 15% |
| JPO-006 | 16% |
| JPO-007 | 3% |
| AnPav498 | 13% |
| JPO-009 | 16% |
| JPO-011 | 15% |
| JPO-012 | 15% |
| JPO-013 | 17% |
| JPO-020 | 20% |
| Name | Relative activity of 80° C./70° C. (%) |
| JPO-001 | 10% |
| JPO-004 | 29% |
| JPO-009 | 13% |
| JPO-014 | 21% |
| JPO-020 | 16% |
| JPO-021 | 30% |
| JPO-052 | 33% |
| Name | Relative activity of 79° C./70° C. (%) |
| JPO-001 | 23% |
| JPO-021 | 46% |
| JPO-022 | 39% |
| JPO-023 | 44% |
| JPO-025 | 51% |
| JPO-027 | 49% |
| JPO-029 | 37% |
| Name | Relative activity of 77° C./70° C. (%) |
| JPO-001 | 72% |
| JPO-029 | 82% |
| JPO-047 | 80% |
| JPO-048 | 90% |
| JPO-049 | 84% |
| JPO-050 | 86% |
| JPO-064 | 87% |
| name | Relative activity of 79° C./77° C. (%) |
| JPO-001 | 36% |
| JPO-029 | 51% |
| JPO-047 | 45% |
| JPO-048 | 81% |
| JPO-049 | 53% |
| JPO-050 | 58% |
| JPO-064 | 65% |
| Name | Relative activity of 79° C./77° C. (%) |
| JPO-001 | 41% |
| JPO-021 | 60% |
| JPO-022 | 48% |
| JPO-023 | 57% |
| JPO-025 | 56% |
| JPO-027 | 64% |
| JPO-029 | 66% |
| JPO-047 | 50% |
| JPO-048 | 72% |
| JPO-051 | 82% |
| JPO-058 | 73% |
| JPO-062 | 72% |
| JPO-063 | 85% |
| JPO-064 | 83% |

TABLE 1b
Lists of the relative activity of PoAMG variants
when compared with their parent JPO-022

| Name | Relative activity of 77° C./70° C. (%) |
|---|---|
| JPO-022 | 60% |
| JPO-027 | 67% |
| JPO-042 | 8% |
| JPO-044 | 86% |
| JPO-045 | 67% |
| JPO-046 | 48% |
| JPO-022 | 76% |
| JPO-023 | 75% |
| JPO-025 | 80% |
| JPO-027 | 84% |
| JPO-058 | 92% |
| JPO-059 | 88% |
| JPO-060 | 86% |
| JPO-061 | 83% |
| JPO-062 | 87% |
| Name | Relative activity of 79° C./77° C. (%) |
| JPO-022 | 49% |
| JPO-023 | 51% |
| JPO-025 | 52% |
| JPO-027 | 58% |
| JPO-058 | 69% |
| JPO-059 | 36% |
| JPO-060 | 41% |
| JPO-061 | 44% |
| JPO-062 | 57% |

TABLE 1c
List of the relative activity of PoAMG variants
when compared with their parent JPO-063

| Name | Relative activity of 79° C./77° C. (%) |
|---|---|
| JPO-063 | 91% |
| JPO-066 | 96% |
| JPO-071 | 89% |
| JPO-072 | 84% |
| JPO-074 | 103% |
| JPO-075 | 86% |
| JPO-076 | 92% |
| JPO-077 | 95% |
| JPO-078 | 88% |
| JPO-079 | 100% |
| Name | Relative activity of 84° C./80° C. (%) |
| JPO-063 | 16% |
| JPO-065 | 26% |
| JPO-067 | 21% |
| JPO-070 | 12% |
| JPO-071 | 13% |
| JPO-074 | 32% |
| JPO-081 | 17% |
| JPO-082 | 24% |
| JPO-083 | 46% |
| JPO-084 | 26% |
| JPO-044 | 37% |
| Name | Relative activity of 82° C./70° C. (%) |
| JPO-063 | 21% |
| JPO-093 | 43% |
| JPO-081 | 25% |
| JPO-088 | 39% |
| JPO-094 | 38% |
| JPO-096 | 38% |
| JPO-106 | 53% |
| Name | Relative activity of 83° C./80° C. (%) |
| JPO-063 | 46% |
| JPO-051 | 44% |
| JPO-096 | 64% |
| JPO-106 | 88% |
| JPO-110 | 81% |
| JPO-111 | 100% |
| JPO-112 | 86% |
| JPO-113 | 83% |
| JPO-114 | 47% |
| JPO-115 | 90% |

TABLE 1d
List of the relative activity of PoAMG variants
when compared with their parent JPO-096

| Name | Relative activity of 83° C./70° C. (%) |
|---|---|
| JPO-082 | 53% |
| JPO-088 | 70% |
| JPO-091 | 69% |
| JPO-092 | 65% |
| JPO-093 | 62% |
| JPO-094 | 74% |
| JPO-095 | 69% |
| JPO-096 | 67% |
| JPO-097 | 65% |
| JPO-098 | 65% |
| Name | Relative activity of 83° C./80° C. (%) |
| JPO-051 | 20% |
| JPO-096 | 43% |
| JPO-109 | 51% |
| JPO-126 | 33% |
| JPO-129 | 48% |
| JPO-130 | 18% |
| JPO-131 | 51% |
| JPO-132 | 34% |

TABLE 1e
List of the relative activity of PoAMG variants
when compared with their parents JPO-129

| Name | Relative activity of 84° C./80° C. (%) |
|---|---|
| JPO-129 | 62% |
| JPO-156 | 51% |
| JPO-160 | 34% |
| JPO-161 | 41% |
| JPO-162 | 49% |
| JPO-163 | 21% |
| JPO-164 | 57% |
| JPO-165 | 77% |

TABLE 1f
List of the relative activity of PoAMG variants
when compared with their parent JPO-166

| Name | Relative activity of 84° C./75° C. (%) |
|---|---|
| JPO-166 | 19% |
| JPO-167 | 66% |
| JPO-168 | 58% |
| JPO-169 | 53% |
| JPO-171 | 47% |
| JPO-172 | 98% |

TABLE 2
Amino acid substitutions in the variants of the PoAMG mature sequence

| Name | Amino acid substitutions |
|---|---|
| PoAMG | The wildtype mature AMG from Penicillium (SEQ ID NO: 1) |
| AMG NL | K79V |
| anPAV498 | P2N P4S P11F T65A K79V Q327F |
| JPO-001 | R1A P2N P4S P11F T65A K79V Q327F |
| JPO-018 | D75N R77D A78Q |
| JPO-019 | D75S R77G A78W V79D F80Y |
| JPO-023 | R1A K34Y S103N |
| JPO-024 | R1A K34Y D445N V447S |
| JPO-025 | R1A K34Y Y504T |
| JPO-026 | R1A S103N D445N V447S |
| JPO-027 | R1A S103N Y504T |
| JPO-028 | R1A D445N V447S Y504T |
| JPO-029 | R1A K34Y S103N D445N V447S |
| JPO-044 | R1A K34Y S103N D445N V447S E501V Y504T |
| JPO-047 | R1A K34Y S103N Y504T |
| JPO-048 | R1A K34Y S103N D445N V447S D566T |
| JPO-049 | R1A K34Y S103N Q594R F595S |
| JPO-050 | R1A K34Y S103N Y504T Q594R F595S |
| JPO-051 | R1A K34Y S103N D445N V447S Y504T Q594R F595S |
| JPO-052 | R1A S105L |
| JPO-053 | R1A S105E |
| JPO-055 | R1A A132R |
| JPO-058 | R1A K34Y S105L Y504T Q594R F595S |
| JPO-059 | R1A K34Y S103N S105L Y504T Q594R F595S |
| JPO-060 | R1A K34Y S103N S105L Y504T Q594R F595S |
| JPO-061 | R1A K34Y S103N S105L Y504T D566T Q594R F595S |
| JPO-062 | R1A K34Y S103N S105L D445N V447S Y504T D566T Q594R F595S |
| JPO-063 | R1A K34Y S103N S105L A132R D445N V447S Y504T D566T Q594R F595S |
| JPO-064 | R1A K34Y S103N S105L D445N V447S D566T Q594R F595S |
| JPO-065 | R1AK34Y S103N S105LA132R D445N V447S E501VY504T D566T Q594R R1A |
| | F595S |
| JPO-066 | R1A K34Y S103N A132R D445N V447S Y504T D566T Q594R F595S |
| JPO-069 | R1A K34Y S103N S105L A132R D445N V447S Y504T D566T V592T |
| JPO-071 | R1A G6S G7T K34Y S103N S105L A132R D445N V447S Y504T D566T Q594R |
| | R1A F595S |
| JPO-074 | R1A K34Y S103N P107L A132R D445N V447S Y504T D566T Q594R F595S |
| JPO-083 | R1A G6S G7T K34Y S103N P107L A132R D445N V447S Y504T D566T Q594R |
| | R1A F595S |
| JPO-084 | R1A G6S G7T K34Y S103N P107L A132R D445N V447S Y504T D566T V592T |
| | R1A Q594R F595S |
| JPO-091 | R1A G6S R7T K34Y S103N P107L A132P D445N V447S Y504T D566T Q594R |
| | F595S |
| JPO-092 | R1A G6S G7T K34Y S103N P107L A132R D445N V447S Y504T D566T T568V |
| | Q594R F595S |
| JPO-093 | R1A G6S G7T K34Y S103N P107L A132P D445N V447S Y504T D566T T568V |
| | Q594R F595S |
| JPO-094 | R1A G6S G7T K34Y S103N P107L A132R D445N V447S S481P Y504T D566T |
| | Q594R F595S |
| JPO-095 | R1A G6S G7T K34Y S103N P107L A132R D445N V447S S481P Y504T D566T |
| | T568V Q594R F595S |
| JPO-096 | R1A G6S G7T K34Y S103N P107L A132P D445N V447S D566T T568V Q594R |
| | F595S |
| JPO-097 | R1A G6S G7T K34Y S103N P107L T110W A132P D445N V447S Y504T D566T |
| | T568V Q594R F595S |
| JPO-098 | R1A G6S G7T K34Y E50R S103N P107L A132P D445N V447S Y504T D566T |
| | T568V Q594R F595S |
| JPO-105 | R1A G6S G7T K34Y S103N P107L A132P D445N V447S E501V Y504T |
| JPO-106 | R1A G6S G7T R31F K34Y S103N P107L A132P D445N V447S Y504T D566T |
| | T568V Q594R F595S |
| JPO-108 | R1A G6S G7T R31F K34Y S103N P107L A132P D445N V447S S481P Y504T |
| | D566T T568V Q594R F595S |
| JPO-109 | R1A G6S G7T K34Y E50R S103N P107L A132P D445N V447S S481P Y504T |
| | D566T T568V Q594R F595S |
| JPO-111 | R1A G6S G7T R31F K34Y S103N P107L A132P D445N V447S S481P E501V |
| | Y504T D566T T568V Q594R F595S |
| JPO-112 | R1A G6S G7T R31F K34Y S103N P107L A132P D445N V447S S481P D566T |
| | T568V Q594R F595S |
| JPO-114 | R1A K34Y D75N R77D A78Q S103N R138L D445N V447S Y504T Q594R F595S |
| JPO-115 | R1A G6S G7T R31F K34Y D75N R77D A78Q S103N P107L A132P D445N |
| | V447S S481P Y504T D566T T568V Q594R F595S |
| JPO-124 | R1A G6S G7T R31F K34Y S103N A132P D445N V447S S481P D566T T568V |
| | Q594R F595S |
| JPO-125 | R1A G6S G7T K34Y E50R S103N A132P D445N V447S S481P D566T T568V |
| | Q594R F595S |
| JPO-126 | R1A R31F K34Y D75N R77D A78Q S103N R138L D445N V447S Y504T Q594R |
| | F595S |
| JPO-127 | R1A K34Y D75N R77D A78Q S103N R138L D445N V447S Q594R F595S |
| JPO-128 | R1A G6S G7T R31F K34Y S103N A132P D445N V447S |
| JPO-129 | R1A G6S G7T R31F K34Y E50R S103N A132P D445N V447S S481P D566T |
| | T568V Q594R F595S |
| JPO-130 | R1A K34Y E50R D75N R77D A78Q S103N R138L D445N V447S Q594R F595S |
| JPO-131 | R1A G6S G7T R31F K34Y E50R D75N R77D A78Q S103N A132P D445N V447S |
| | S481P D566T Q594R F595S |
| JPO-132 | R1A R31F K34Y E50R D75N R77D A78Q S103N R138L D445N V447S Q594R |
| | F595S |
| JPO-133 | R1A G6S G7T R31F K34Y E50R D75N R77D A78Q S103N A132P R138L D445N |
| | V447S S481P D566T Q594R F595S |
| JPO-138 | R1A R135S |
| JPO-143 | R1A G6S G7T R31F K34Y E50R S103N A132P D445N V447S S481P E501L |
| | D566T T568V Q594R F595S |
| JPO-154 | R1A G6S G7T R31F K34Y S103N A132P R138G D445N V447S S481P D566T |
| | T568V Q594R F595S |
| JPO-155 | R1A G6S G7T R31F K34Y S103N A132P R138L D445N V447S S481P D566T |
| | T568V Q594R F595S |
| JPO-156 | R1A G6S G7T R31F K34Y S103N A132P R138P D445N V447S S481P D566T |
| | T568V Q594R F595S |
| JPO-167 | R1A G6S G7T R31F K34Y E50R S103N A132P S379P D445N V447S S481P |
| | E501A D566T T568V Q594R F595S |
| JPO-168 | R1A G6S G7T R31F K34Y E50R S103N A132P D445N V447S S481P T484P |
| | E501A D566T T568V Q594R F595S |
| JPO-169 | R1A G6S G7T R31F K34Y E50R S103N A132P D445N V447S S481P E501A |
| | N539P D566T T568V Q594R F595S |
| JPO-171 | R1A G6S G7T R31F K34Y E50R S103N A132P S379P D445N V447S S481P |
| | T484P E501A D566T T568V Q594R F595S |
| JPO-172 | R1A G6S G7T R31F K34Y E50R S103N A132P D445N V447S S481P T484P |
| | E501A N539P D566T T568V Q594R F595S |

### Example 3: Fermentation of the Aspergillus niger

Aspergillus niger strains were fermented on a rotary shaking table in 500 ml baffled flasks containing 100 ml MU1 with 4 ml 50% urea at 220 rpm, 30° C. The culture broth was centrifuged (10,000×g, 20 min) and the supernatant was carefully decanted from the precipitates.

### Example 4: Purification of PoAMG (JPO-001) Variants

PoAMG variants were purified by cation exchange chromatography. The peak fractions of each were pooled individually and dialyzed against 20 mM sodium acetate buffer pH 5.0, and then the samples were concentrated using a centrifugal filter unit (Vivaspin Turbo 15, Sartorius). Enzyme concentrations were determined by A280 value.

### Example 5: Thermostability Determination (TSA)

Purified enzyme was diluted with 50 mM sodium acetate buffer pH 5.0 to 0.5 mg/ml and mixed with equal volume of SYPRO Orange (Invitrogen) diluted with Milli-Q water. Eighteen ul of mixture solution were transfer to LightCycler 480 Multiwell Plate 384 (Roche Diagnostics) and the plate was sealed.

### Equipment Parameters of TSA:

Apparatus: LightCycler 480 Real-Time PCR System (Roche Applied Science)
Scan rate: 0.02° C./sec
Scan range: 37-96° C.
Integration time: 1.0 sec
Excitation wave length 465 nm
Emission wave length 580 nm

The obtained fluorescence signal was normalized into a range of 0 and 1. The Td was defined as the temperature at which the signal intensity was 0.5. The thermostability improvements are listed in Table 3 with Td of the PoAMG variant denoted anPAV498 as 0.

### Example 6: PoAMG Activity Assay

Maltodextrin (DE11) assay by GOD-POD method

### Substrate Solution

30 g maltodextrin (pindex #2 from MATSUTANI chemical industry Co., Ltd.)
100 ml 120 mM sodium acetate buffer, pH 5.0
   Glucose CII test kit (Wako Pure Chemical Industries, Ltd.)

Twenty ul of enzyme samples were mixed with 100 ul of substrate solution and incubated at set temperatures for 2 hours. The samples were cooled down on the aluminum block for 3 min then 10 ul of the reaction solution was mixed with 590 ul of 1 M Tris-HCl PH 8.0 to stop reaction. Ten ul of the solution was mixed with 200 ul of the working solution of the test kit then stand at room temperature for 15 min. The absorbance at A505 was read. The activities are listed in Table 3 as relative activity of the PoAMG variant denoted anPAV498.

TABLE 3

| | Td improvement [° C.] | Activity at 91° C. |
|---|---|---|
| Variant | (pH5.0, anPAV498 as 0) | (anPAV498 as 100) |
| anPAV498 | — | 100 |
| JPO-001 | 1.0 | 94 |
| JPO-004 | 2.2 | — |
| JPO-009 | 0.7 | — |
| JPO-013 | 1.5 | — |
| JPO-014 | 2.3 | — |
| JPO-020 | 1.4 | 74 |
| JPO-021 | 2.5 | 113 |
| JPO-052 | 2.6 | 85 |
| JPO-053 | 0.2 | 71 |
| JPO-055 | 1.6 | 85 |
| JPO-023 | 3.6 | — |
| JPO-024 | 2.5 | — |
| JPO-025 | 3.4 | — |
| JPO-027 | 2.9 | — |
| JPO-029 | 3.7 | 191 |
| JPO-048 | 4.3 | 163 |
| JPO-051 | 5.7 | 222 |
| JPO-058 | 4.2 | 157 |
| JPO-062 | 4.2 | 159 |
| JPO-063 | 5.4 | 107 |
| JPO-064 | 4.9 | 178 |
| JPO-065 | 7.0 | 127 |
| JPO-066 | 6.5 | 178 |
| JPO-069 | 4.8 | 95 |
| JPO-071 | 6.1 | 128 |
| JPO-074 | 6.3 | 108 |
| JPO-081 | 5.5 | 213 |
| JPO-082 | 5.6 | 215 |
| JPO-089 | 6.0 | 171 |
| JPO-090 | 5.5 | 155 |
| JPO-018 | 0.6 | 84 |
| JPO-019 | 0.5 | 86 |
| JPO-044 | 6.3 | 225 |
| JPO-083 | 6.1 | 103 |
| JPO-084 | 4.4 | 66 |
| JPO-099 | 6.8 | 156 |
| JPO-091 | 6.6 | 130 |
| JPO-092 | 6.7 | 113 |
| JPO-093 | 6.8 | 132 |
| JPO-094 | 6.6 | 126 |
| JPO-095 | 6.9 | — |
| JPO-096 | 5.9 | — |
| JPO-097 | 5.2 | — |
| JPO-098 | 5.6 | — |
| JPO-112 | 8.2 | — |
| JPO-114 | 5.2 | 218 |
| JPO-115 | 8.0 | — |
| JPO-108 | 8.5 | — |
| JPO-109 | 7.2 | — |
| JPO-111 | 8.4 | — |
| JPO-124 | 8.0 | 385 |
| JPO-125 | 6.8 | 324 |
| JPO-126 | 6.6 | 268 |
| JPO-127 | 4.9 | 246 |
| JPO-129 | 8.2 | 399 |
| JPO-130 | 5.3 | 278 |
| JPO-131 | 7.9 | 367 |
| JPO-132 | 6.6 | 336 |
| JPO-138 | 6.4 | 125 |
| JPO-133 | 6.1 | 143 |
| JPO-143 | 8.8 | 280 |
| JPO-154 | 7.6 | 252 |
| JPO-155 | 8.3 | 282 |
| JPO-156 | 8.3 | 290 |
| JPO-145 | 8.2 | — |
| JPO-147 | 8.2 | — |
| JPO-150 | 8.2 | — |
| JPO-152 | 8.4 | — |
| JPO-153 | 9.0 | 399 |
| JPO-161 | 6.0 | 200 |
| JPO-165 | 8.9 | 403 |
| JPO-166 | 7.0 | 237 |
| JPO-167 | 9.1 | 387 |
| JPO-168 | 9.3 | 332 |
| JPO-169 | 9.6 | 269 |
| JPO-171 | 9.4 | 255 |
| JPO-172 | 9.9 | 432 |

### Example 7: Freshness Effect of AMG in Bread (Part 1)

Bread was baked in a straight dough process with a recipe according to Table 4. The bread was baked in lidded tins in order to have the same volume of all bread. The ingredients were mixed in a spiral mixer into a dough for 3+7 min at 17 respectively 35 rpm. The doughs were divided into 450 g pieces, rounded, sheeted and place in baking tins. The tins with the doughs were proofed for 55 min at 32° C. and 86% relative humidity. The proofed doughs were baked in a deck oven for 35 min at 230° C.

TABLE 4

| | Bakers % |
|---|---|
| Flour (Kolibri, Meneba, NL) | 100 |
| Water | 55.5 |
| Yeast | 4.5 |
| Sucrose | 1.5 |
| Salt | 1.5 |
| Ascorbic acid | 0.04 |
| Calcium Propionate | 0.25 |
| Fungamyl ® 4000SG | 6 ppm |
| Panzea ® BG | 25 ppm |

TABLE 5
Seven dough treatments were prepared with different enzymatic
additions according to Table 4; AMG Goldcrust ® 3300
BG (Goldcrust ®) is a commercially available AMG
for baking (Novozymes A/S, Denmark); AMG NL and AMG anPAV498
are artificial variants of PoAMG (see table 2).

| Dough | | | | | | |
|---|---|---|---|---|---|---|
| 1 | 2 | 3 | 4 | 5 | 6 | 7 |

| | | | | | | |
|---|---|---|---|---|---|---|
| AMG Goldcrust ®, | | | 25 | 50 | | |
| mgEP/kg flour | | | | | | |
| AMG NL, | | 25 | | | 50 | |
| mgEP/kg flour | | | | | | |
| AMG anPAV498, | 25 | | | | | 50 |
| mgEP/kg flour | | | | | | |

The doughs were baked and the resulting breads were packed 2 hours after baking in sealed plastic bags and stored at room temperature until analysis.

The texture of each bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product. A standard method for measuring firmness and elasticity is based on force-deformation of the baked product. A force-deformation of the baked products may be performed with a 40 mm diameter cylindrical probe. The force on the cylindrical probe is recorded as it is pressed down 40% strain a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 40% strain (corresponding to force at time=40 s for a bread slice of 25 mm thickness) divided by the force needed to press the probe 10 mm into the crumb (corresponding to force at time=10 s for a bread slice of 25 mm thickness) times 100.

The results from the texture analysis can be found in Table 6 (firmness) and Table 7 (elasticity).

Fresh bread without enzyme (control) has low firmness and high elasticity, as the bread is stored the firmness increase over time and the elasticity decrease. Traditional AMGs used in baking applications (for example Goldcrust) does not impact the Firmness or elasticity.

AMG anPAV498 dosed at 25 or 50 mgEP/kg as well as AMG NL dosed at 50 mgEP/kg flour improves (decrease) the initial firmness and reduces the increase in firmness over time. AMG anPAV498 dosed at 25 or 50 mgEP/kg as well as AMG NL dosed at 50 mgEP/kg flour improves (increase) the initial elasticity and prevents the loss of elasticity over time.

TABLE 6
Firmness (g) on day 1, 3 and 7 of bread
with enzyme treatments according Table 5

| Day | 1 | 3 | 7 |
|---|---|---|---|
| Control | 411 | 690 | 1025 |
| 25 mgEP/kg AMG anPAV498 | 332 | 446 | 746 |
| 25 mgEP/kg AMG NL | 413 | 689 | 1019 |
| 25 mgEP/kg Goldcrust ® | 406 | 709 | 1094 |
| 50 mgEP/kg Goldcrust ® | 417 | 736 | 1071 |
| 50 mgEP/kg AMG NL | 388 | 579 | 875 |
| 50 mgEP/kg AMG anPAV498 | 314 | 426 | 662 |

TABLE 7
Elasticity (%) on day 1, 3 and 7 of bread
with enzyme treatments according Table 5

| Day | 1 | 3 | 7 |
|---|---|---|---|
| Control | 61.0 | 54.0 | 48.0 |
| 25 mgEP/kg AMG anPAV498 | 63.3 | 59.6 | 53.9 |
| 25 mgEP/kg AMG NL | 60.7 | 54.9 | 49.3 |
| 25 mgEP/kg Goldcrust ® | 60.4 | 54.7 | 48.3 |
| 50 mgEP/kg Goldcrust ® | 60.6 | 53.4 | 48.4 |
| 50 mgEP/kg AMG NL | 61.9 | 56.9 | 51.1 |
| 50 mgEP/kg AMG anPAV498 | 66.1 | 63.2 | 57.1 |

Sugars were extracted from the bread crumb using 0.1 M Phosphate buffer pH 8.0 in 70% EtOH. Bread crumb (180 mg) were added to the extraction buffer (1.8 ml) and was incubated for 20 minutes at 70° C. during mixing. The bread crumb was spun down at 12,000 rpm for 5 minutes in a centrifuge and 500 μl of the supernatant was taken and diluted 200× using a 20 mM Phosphate buffer pH 8.0+10 mg/L cellobiose as internal standard. The extracted sugars (glucose, fructose, maltose and maltotriose) were quantified on an ICS-5000 HPLC system with a CarboPac PA1 column. A theoretical sweetness was calculated based on the levels of glucose, fructose and maltose was calculated using sweetness intensity factors. The sweetness factors in Table 8 was based on the determinations in Portmann M O, Birch G. J Sci Food Agric 69(3):275-81, 1995.

TABLE 8

| Sugar | Sweetness intensity factor |
|---|---|
| Glucose | 0.5 |
| Fructose | 1 |
| Maltose | 0.2 |

The amount of simple sugars (glucose fructose, maltose and maltotriose) can be found in Table 9 along with a theoretical sweetness calculated on the amount of the individual sugars. All three AMGs increase the amount of simple sugars. Both AMG NL and AMG anPAV498 are more efficient in generating glucose compared to Goldcrust® resulting in a higher theoretical sweetness.

TABLE 9
Amount of sugars (g/kg bread crumb) extracted from
dough treated with enzymes according to Table 5

| | | fruc- | | | Sweet- |
|---|---|---|---|---|---|
| | glucose | tose | maltose | maltotriose | ness |
| Control | 0.7 | 3.4 | 17.0 | 1.0 | 7.2 |
| 25 mgEP AMG | 15.1 | 4.2 | 14.8 | 0.6 | 14.7 |
| anPAV498 | | | | | |
| 25 mgEP AMG NL | 7.6 | 4.7 | 14.0 | 0.5 | 11.3 |
| 25 mgEP Goldcrust ® | 3.4 | 4.6 | 13.4 | 0.5 | 9.0 |
| 50 mgEP Goldcrust ® | 7.2 | 5.3 | 13.8 | 0.5 | 11.6 |
| 50 mgEP AMG NL | 14.5 | 5.2 | 13.7 | 0.4 | 15.2 |
| 50 mgEP AMG | 31.1 | 5.1 | 14.1 | 0.5 | 23.4 |
| anPAV498 | | | | | |

### Example 8. Freshness Effect of AMG (part 2)

Bread was baked in a straight dough process with a recipe according to

Table 10. The bread was baked in lidded tins in order to have the same volume of all bread. The ingredients were mixed in a spiral mixer into a dough for 3+7 min at 17 respectively 35 rpm. The doughs were divided into 450 g pieces, rounded, sheeted and place in baking tins. The tins with the doughs were proofed for 55 min at 32° C. and 86% relative humidity. The proofed doughs were baked in a deck oven for 35 min at 230° C.

TABLE 10

| | Bakers % |
|---|---|
| Flour (Kolibri, Meneba, NL) | 100 |
| Water | 55.5 |
| Yeast | 4.5 |
| Sucrose | 1.5 |
| Salt | 1.5 |
| Ascorbic acid | 0.04 |
| Calcium Propionate | 0.25 |
| Fungamyl ® 4000SG | 6 ppm |
| Panzea ® BG | 25 ppm |

TABLE 11
Seven dough treatments were prepared
with different enzymatic additions

Dough

| | | | | | | |
|---|---|---|---|---|---|---|
| 1 | 2 | 3 | 4 | 5 | 6 | 7 |

| | | | | | | |
|---|---|---|---|---|---|---|
| AMG anPAV498, | | | 25 | 50 | | |
| mgEP/kg flour | | | | | | |
| JPO124, | | 25 | | | 50 | |
| mgEP/kg flour | | | | | | |
| JPO172, | 25 | | | | | 50 |
| mgEP/kg flour | | | | | | |

The doughs were baked and the resulting breads were packed 2 hours after baking in sealed plastic bags and stored at room temperature until analysis.

The texture of each bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product.

A standard method for measuring firmness and elasticity is based on force-deformation of the baked product. A force-deformation of the baked products may be performed with a 40 mm diameter cylindrical probe. The force on the cylindrical probe is recorded as it is pressed down 40% strain on a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 40% strain (corresponding to force at time=40 s for a bread slice of 25 mm thickness) divided by the force needed to press the probe 10 mm into the crumb (corresponding to force at time=10 s for a bread slice of 25 mm thickness) times 100.

The results from the texture analysis can be found in Table 12 (firmness) and Table 13 (elasticity).

Fresh bread without enzyme (control) has low firmness and high elasticity, as the bread is stored the firmness increase over time and the elasticity decrease. Traditional AMGs used in baking applications (for example Goldcrust) does not impact the firmness or elasticity (Example 7).

All three AMGs (AMG anPAV498, JPO124 and JPO172) dosed at 25 or 50 mgEP/kg improved (decreased) the initial firmness and reduced the increase in firmness over time. All three AMGs (AMG anPAV498, JPO124 and JPO172) dosed at 25 or 50 mgEP/kg improved (increased) the initial elasticity and prevented the loss of elasticity over time.

TABLE 12
Firmness (g) on day 1, 3 and 7 of bread with
enzyme treatments according Table 11.

| Treatment | Day 1 | Day 3 | Day 7 |
|---|---|---|---|
| Control | 519 | 687 | 883 |
| 25 mgEP/kg AMG anPAV498 | 377 | 513 | 716 |
| 50 mgEP/kg AMG anPAV498 | 337 | 434 | 651 |
| 25 mgEP/kg JPO124 | 311 | 400 | 612 |
| 50 mgEP/kg JPO124 | 288 | 313 | 516 |
| 25 mgEP/kg JPO172 | 313 | 351 | 502 |
| 50 mgEP/kg JPO172 | 240 | 289 | 433 |

TABLE 13
Elasticity (%) on day 1, 3 and 7 of bread
with enzyme treatments according Table 11.

| Treatment | Day 1 | Day 3 | Day 7 |
|---|---|---|---|
| Control | 59.5 | 51.6 | 47.1 |
| 25 mgEP/kg AMG anPAV498 | 63.5 | 58.5 | 51.3 |
| 50 mgEP/kg AMG anPAV498 | 66.5 | 62.2 | 55.9 |
| 25 mgEP/kg JPO124 | 65.9 | 62.8 | 56.5 |
| 50 mgEP/kg JPO124 | 66.9 | 66.7 | 61.2 |
| 25 mgEP/kg JPO172 | 66.4 | 64.0 | 58.7 |
| 50 mgEP/kg JPO172 | 69.1 | 67.8 | 64.4 |

Sugars were extracted from the bread crumb using 0.1 M Phosphate buffer pH 8.0 in 70% EtOH. Bread crumb (180 mg) were added to the extraction buffer (1.8 ml) and was incubated for 20 minutes at 70° C. during mixing. The bread crumb was spun down at 12,000 rpm for 5 minutes in a centrifuge and 500 μl of the supernatant was taken and diluted 200× using a 20 mM Phosphate buffer pH 8.0+10 mg/L cellobiose as internal standard. The extracted sugars (glucose, fructose, maltose and maltotriose) were quantified on an ICS-5000 HPLC system with a CarboPac PA1 column. A theoretical sweetness was calculated based on the levels of glucose, fructose and maltose was calculated using sweetness intensity factors. The sweetness factors in

Table 13 were based on the determinations in Portmann M O, Birch G. J Sci Food Agric 69(3):275-81, 1995.

TABLE 14

| Sugar | Sweetness intensity factor |
|---|---|
| Glucose | 0.5 |
| Fructose | 1 |
| Maltose | 0.2 |

The amount of simple sugars (glucose fructose, maltose and maltotriose) can be found in Table 1 along with a theoretical sweetness calculated on the amount of the individual sugars. All three AMGs increase the amount of simple sugars and increased the calculated sweetness. The higher dosage of the AMGs the more glucose was generated and higher theoretical sweetness. JPO0172 and JPO124 were more efficient than AMG anPAV498 in increasing the glucose and the theoretical sweetness.

TABLE 15
Simple sugars (g/kg bread crumb) extracted from
bread treated with enzymes according to Table 11.

| | | Fruc- | | | Sweet- |
|---|---|---|---|---|---|
| | Glucose | tose | Maltose | Maltotriose | ness |
| Control | 1.5 | 6.1 | 27.5 | 1.4 | 12 |
| 25 mgEP/kg JPO172 | 39.3 | 7.7 | 24.6 | 0.7 | 32 |
| 25 mgEP/kg JPO124 | 38.6 | 9.7 | 28.1 | 0.8 | 35 |
| 25 mgEP/kg AMG | 24.0 | 8.3 | 24.8 | 0.8 | 25 |
| anPAV498 | | | | | |
| 50 mgEP/kg AMG | 48.0 | 10.2 | 23.2 | 0.6 | 39 |
| anPAV498 | | | | | |
| 50 mgEP/kg JPO124 | 62.3 | 10.6 | 22.1 | 0.5 | 46 |
| 50 mgEP/kg JPO172 | 68.6 | 10.8 | 21.7 | 0.5 | 49 |

The change in sugar levels in the bread crumb as a function of bread storage time at ambient temperature can be found in Tables 16-20. The glucose level table 16 which is the product of the AMG is stable over bread storage time. The same picture is seen for the other sugars extracted from the bread crumb fructose (table 17), maltose (table 18), maltotriose (table 19) and Maltotetraose (table 20)

TABLE 16
Glucose levels (g/kg bread crumb) in bread crumb
over time as a function of enzyme treatment.

| Glucose | | Day 0 | Day 3 | Day 7 |
|---|---|---|---|---|
| AMG anPAV498 | 0 | 1.5 | 1.4 | 1.5 |
| | 25 | 23.9 | 24.9 | 24.3 |
| | 50 | 48.0 | 45.3 | 46.9 |
| JP0124 | 0 | 1.5 | 1.4 | 1.5 |
| | 25 | 38.6 | 37.0 | 36.9 |
| | 50 | 62.3 | 62.4 | 68.0 |
| JP0172 | 0 | 1.5 | 1.4 | 1.5 |
| | 25 | 39.3 | 38.6 | 40.7 |
| | 50 | 68.6 | 72.0 | 74.0 |

TABLE 17
Maltose levels (g/kg bread crumb) in bread crumb
over time as a function of enzyme treatment.

| Maltose | | Day 0 | Day 3 | Day 7 |
|---|---|---|---|---|
| AMG ANPAV498anPAV498 | 0 | 27.5 | 28.0 | 24.4 |
| | 25 | 24.8 | 25.4 | 25.5 |
| | 50 | 23.2 | 22.5 | 23.0 |
| JP0124 | 0 | 27.5 | 28.0 | 24.4 |
| | 25 | 28.1 | 25.1 | 24.8 |
| | 50 | 22.1 | 19.6 | 21.5 |
| JP0172 | 0 | 27.5 | 28.0 | 24.4 |
| | 25 | 24.6 | 24.8 | 24.7 |
| | 50 | 21.7 | 21.2 | 21.2 |

TABLE 18
Fructose levels (g/kg bread crumb) in bread crumb
over time as a function of enzyme treatment.

| Fructose | | Day 0 | Day 3 | Day 7 |
|---|---|---|---|---|
| AMG ANPAV498 | 0 | 6.1 | 6.5 | 6.7 |
| | 25 | 8.3 | 8.7 | 9.0 |
| | 50 | 10.2 | 10.1 | 10.4 |
| JP0124 | 0 | 6.1 | 6.5 | 6.7 |
| | 25 | 9.7 | 8.9 | 9.2 |
| | 50 | 10.6 | 10.6 | 10.4 |
| JP0172 | 0 | 6.1 | 6.5 | 6.7 |
| | 25 | 7.7 | 8.1 | 8.5 |
| | 50 | 10.8 | 10.4 | 11.3 |

TABLE 19
Maltotriose levels (g/kg bread crumb) in bread crumb
over time as a function of enzyme treatment.

| Maltotriose | | Day 0 | Day 3 | Day 7 |
|---|---|---|---|---|
| AMG ANPAV498 | 0 | 1.4 | 1.3 | 1.2 |
| | 25 | 0.8 | 0.7 | 0.8 |
| | 50 | 0.6 | 0.6 | 0.6 |
| JP0124 | 0 | 1.4 | 1.3 | 1.2 |
| | 25 | 0.8 | 0.7 | 0.7 |
| | 50 | 0.5 | 0.5 | 0.6 |
| JP0172 | 0 | 1.4 | 1.3 | 1.2 |
| | 25 | 0.7 | 0.7 | 0.7 |
| | 50 | 0.5 | 0.5 | 0.5 |

TABLE 20
Maltotetraose levels (g/kg bread crumb) in bread crumb
over time as a function of enzyme treatment.

| Maltotetraose | | Day 0 | Day 3 | Day 7 |
|---|---|---|---|---|
| AMG ANPAV498 | 0 | 0.5 | 0.4 | 0.4 |
| | 25 | 0.4 | 0.3 | 0.4 |
| | 50 | 0.3 | 0.3 | 0.2 |
| JP0124 | 0 | 0.5 | 0.4 | 0.4 |
| | 25 | 0.3 | 0.3 | 0.3 |
| | 50 | 0.2 | 0.2 | 0.2 |
| JP0172 | 0 | 0.5 | 0.4 | 0.4 |
| | 25 | 0.3 | 0.3 | 0.3 |
| | 50 | 0.2 | 0.2 | 0.2 |

### Example 9. Freshness Effect of AMG in Combination with Novamyl

Bread was baked in a straight dough process with a recipe according to table 21. The bread was baked in lidded tins in order to have the same volume of all bread. The ingredients were mixed in a spiral mixer into a dough for 3+7 min at 17 respectively 35 rpm. The doughs were divided into 450 g pieces, rounded, sheeted and place in baking tins. The tins with the doughs were proofed for 55 min at 32° C. and 86% relative humidity. The proofed doughs were baked in a deck oven for 35 min at 230° C.

TABLE 21

| | Bakers % |
|---|---|
| Flour (Kolibri, Meneba, NL) | 100 |
| Water | 55.5 |
| Yeast | 4.5 |
| Sucrose | 1.5 |
| Salt | 1.5 |
| Ascorbic acid | 0.04 |
| Calcium Propionate | 0.25 |
| Fungamyl 4000SG | 6 ppm |
| Panzea BG | 25 ppm |

TABLE 22
Seven treatments were prepared with different enzymatic additions

| | Dough | | | | | | |
|---|---|---|---|---|---|---|---|
| | 1 | 2 | 3 | 4 | 5 | 6 | 7 |
| AMG anPAV498, | 25 | 50 | 25 | — | 25 | 50 | — |
| mgEP/kg flour | | | | | | | |
| Novamyl ® 3D, ppm | — | 30 | 15 | — | 30 | — | 30 |

The bread was packed 2 hours after baking in sealed plastic bags and stored at room temperature until analysis.

The texture of each bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product. A standard method for measuring firmness and elasticity is based on force-deformation of the baked product. A force-deformation of the baked products may be performed with a 40 mm diameter cylindrical probe. The force on the cylindrical probe is recorded as it is pressed down 40% strain on a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness). Elasticity (in %) is defined as the force recoded after 30 seconds compression at 40% strain (corresponding to force at time=40 s for a bread slice of 25 mm thickness) divided by the force needed to press the probe 10 mm into the crumb (corresponding to force at time=10 s for a bread slice of 25 mm thickness) times 100.

The results from the texture analysis can be found in table 23 (firmness) and table 24 (elasticity). Fresh bread without enzyme (Control) has low firmness and high elasticity, as the bread is stored the firmness increase over time and the elasticity decrease.

AMG anPAV498 improves the initial firmness and elasticity as well as reduce the changes in firmness and elasticity over time.

Novamyl® 3D does not impact the initial firmness or elasticity. However, Novamyl® 3D reduces the change in firmness and elasticity over time.

The combination of AMG anPAV498 and Novamyl® 3D both improves the initial firmness and elasticity compared to no enzyme or Novamyl® 3D alone, as well as well as the change in firmness and elasticity over time. The combination results in a bread with the best firmness and elasticity after 7 days of storage.

TABLE 23
Firmness (g) on day 1, 3 and 7 of bread with
enzyme treatments according Table 22.

| Treatment | Day 1 | Day 7 | Day 14 |
|---|---|---|---|
| 25 mgEP/kg flour AMG anPAV498 | 321 | 446 | 741 |
| 50 mgEP/kg flour AMG | 506 | 552 | 704 |
| anPAV498 + 30 ppm Novamyl ® 3D | | | |
| 25 mgEP/kg flour AMG | 344 | 544 | 668 |
| anPAV498 + 15 ppm Novamyl ® 3D | | | |
| Control | 499 | 733 | 1099 |
| 25 mgEP/kg flour AMG | 407 | 512 | 579 |
| anPAV498 + 30 ppm Novamyl ® 3D | | | |
| 50 mgEP/kg flour AMG anPAV498 | 317 | 401 | 769 |
| 30 ppm Novamyl ® 3D | 480 | 607 | 769 |

TABLE 24
Elasticity (%) on day 1, 3 and 7 of bread
with enzyme treatments according Table 22.

| Treatment | Day 1 | Day 7 | Day 14 |
|---|---|---|---|
| 25 mgEP/kg flour AMG anPAV498 | 65.9 | 60.8 | 50.5 |
| 50 mgEP/kg flour AMG | 61.8 | 61.5 | 58.4 |
| anPAV498 + 30 ppm Novamyl ® 3D | | | |
| 25 mgEP/kg flour AMG | 64.1 | 61.4 | 58.2 |
| anPAV498 + 15 ppm Novamyl ® 3D | | | |
| Control | 60.6 | 55.2 | 46.4 |
| 25 mgEP/kg flour AMG | 61.2 | 59.5 | 58.0 |
| anPAV498 + 30 ppm Novamyl ® 3D | | | |
| 50 mgEP/kg flour AMG anPAV498 | 67.3 | 64.4 | 56.1 |
| 30 ppm Novamyl ® 3D | 60.2 | 58.4 | 55.0 |

Sugars were extracted from the bread crumb using 0.1 M Phosphate buffer pH 8.0 in 70% EtOH. Bread crumb (180 mg) were added to the extraction buffer (1.8 ml) and was incubated for 20 minutes at 70° C. during mixing. The bread crumb was spun down at 12,000 rpm for 5 minutes in a centrifuge and 500 μl of the supernatant was taken and diluted 200× using a 20 mM Phosphate buffer pH 8.0+10 mg/L cellobiose as internal standard. The extracted sugars (glucose, fructose, maltose and maltotriose) were quantified on an ICS-5000 HPLC system with a CarboPac PA1 column. A theoretical sweetness was calculated based on the levels of glucose, fructose and maltose was calculated using sweetness intensity factors. The sweetness factors in

Table 25 were based on the determinations in Portmann M O, Birch G. J Sci Food Agric 69(3):275-81, 1995.

TABLE 25

| Sugar | Sweetness intensity factor |
|---|---|
| Glucose | 0.5 |
| Fructose | 1 |
| Maltose | 0.2 |

The amounts of different sugars extracted from the bread and the theoretically calculated sweetness based on the sugar amounts can be found in Table 26. The higher dosage of AMG anPAV498 the more glucose in the bread. The higher dosage of Novamyl® 3D the more maltose and maltotriose in the dough. The combination of AMG anPAV498 and Novamyl® 3D increase both glucose, maltose and maltotriose. The main contributor to the calculated sweetness is the dose of AMG anPAV498 since glucose impacts sweetness more than maltose and maltotriose.

TABLE 26
Simple sugars (g/kg bread crumb) extracted from
bread treated with enzymes according to Table 22.

| | Glucose | Fructose | Maltose | Maltotriose | Sweetness |
|---|---|---|---|---|---|
| 25 mgEP/kg AMG anPAV498 | 17.4 | 4.6 | 17.2 | 0.6 | 17 |
| 50 mgEP/kg AMG anPAV498 + | 33.4 | 7.0 | 24.5 | 12.7 | 29 |
| 30 ppm Novamyl ® 3D | | | | | |
| 25 mgEP/kg AMG anPAV498 + | 18.0 | 7.1 | 21.9 | 6.8 | 20 |
| 15 ppm flour Novamyl ® 3D | | | | | |
| Control | 1.0 | 4.0 | 20.3 | 1.0 | 9 |
| 25 mgEP/kg AMG anPAV498 + | 15.6 | 5.4 | 29.1 | 9.6 | 19 |
| 30 ppm Novamyl ® 3D | | | | | |
| 50 mgEP/kg AMG anPAV498 | 34.9 | 6.3 | 17.1 | 0.5 | 27 |
| 30 ppm Novamyl ® 3D | 2.3 | 3.5 | 32.8 | 5.2 | 11 |

### Example 10. Dosage Response of AMG NL (Partial Sugar Replacement)

Bread was baked in a straight dough process with a recipe according to Table 27. The ingredients were mixed in a spiral mixer into a dough for 3+7 min at 17 respectively 35 rpm. The doughs were divided into 450 g pieces, rounded, sheeted and place in baking tins. The tins with the doughs were proofed for 55 min at 32° C. and 86% relative humidity. The proofed doughs were baked in a deck oven for 25 min at 230° C.

TABLE 27
Fungamyl ® 4000SG is a commercially available
fungal amylase for baking (Novozymes A/S, Denmark),
Panzea ® BG is a commercially available
bacterial xylanase for baking (Novozymes A/S, Denmark).

| | Bakers % |
|---|---|
| Flour (Kolibri, Meneba, NL) | 100 |
| Water | 55.5 |
| Yeast | 3 |
| Sucrose | 5 |
| Salt | 0.5 |
| Ascorbic acid | 0.04 |
| Calcium Propionate | 0.25 |
| Fungamyl ® 4000SG | 7 ppm |
| Panzea ® BG | 25 ppm |

TABLE 28
Eight treatments were prepared with different enzymatic
additions, AMG Goldcrust ® is a commercially
available AMG for baking (Novozymes A/S, Denmark) and
JA126 is a raw-starch degrading amylase (Novozymes A/S, Denmark).

| | Dough | | | | | | | |
|---|---|---|---|---|---|---|---|---|
| | 1 | 2 | 3 | 4 | 5 | 6 | 7 | 8 |
| AMG | 112.5 | — | — | — | — | — | — | — |
| Goldcrust ® | | | | | | | | |
| mgEP/kg | | | | | | | | |
| flour | | | | | | | | |
| AMG NL, | | 17.9 | 26.8 | 35.7 | 44.6 | 53.6 | 62.5 | 71.4 |
| mgEP/kg | | | | | | | | |
| flour | | | | | | | | |
| JA126, | 0.35 | 0.35 | 0.35 | 0.35 | 0.35 | 0.35 | 0.35 | 0.35 |
| mgEP/kg | | | | | | | | |
| flour | | | | | | | | |

The dough properties were evaluated by a trained baker and the volume of the bread was determined using Volscan profiler (Stable microsystems, Godalming, UK). The results from the evaluation can be found in

Table 29. The doughs with 44.6 and 53.6 mgEP/kg flour of AMG NL (Doughs 5 & 6) had similar volume, the same extensibility and elasticity as the dough with 112.5 mgEP/kg flour of AMG Goldcrust®, but the doughs were slightly less sticky and soft.

TABLE 29

| | Dough | | | | | | | |
|---|---|---|---|---|---|---|---|---|
| | 1 | 2 | 3 | 4 | 5 | 6 | 7 | 8 |
| Stickiness | 5 | 4 | 4 | 4 | 4 | 4 | 4 | 4 |
| Softness | 5 | 4 | 4 | 4 | 4 | 4 | 4 | 4 |
| Extensibility | 5 | 3 | 3 | 3 | 5 | 5 | 5 | 5 |
| Elasticity | 5 | 6 | 6 | 6 | 5 | 5 | 5 | 5 |
| Spec vol ml/g | 4.4 | 4.3 | 4.2 | 4.2 | 4.3 | 4.2 | 4.1 | 4.1 |

Sugars were extracted from the bread crumb using 0.1 M Phosphate buffer pH 8.0 in 70% EtOH. Bread crumb (180 mg) were added to the extraction buffer (1.8 ml) and was incubated for 20 minutes at 70° C. during mixing. The bread crumb was spun down at 12,000 rpm for 5 minutes in a centrifuge and 500 μl of the supernatant was taken and diluted 200× using a 20 mM Phosphate buffer pH 8.0+10 mg/L cellobiose as internal standard. The extracted sugars (glucose, fructose, maltose and maltotriose) were quantified on an ICS-5000 HPLC system with a CarboPac PA1 column. A theoretical sweetness was calculated based on the levels of glucose, fructose and maltose was calculated using sweetness intensity factors. The sweetness factors in Table 30 were based on the determinations in Portmann M O, Birch G. J Sci Food Agric 69(3):275-81, 1995.

TABLE 30

| Sugar | Sweetness intensity factor |
|---|---|
| Glucose | 0.5 |
| Fructose | 1 |
| Maltose | 0.2 |

The amounts of sugars (g/kg bread crumb) and theoretical sweetnesses can be found in Table 31. Based on these sugar levels it can be calculated that a dough with 44.6 mg enzyme protein (mgEP) per kg flour of AMG NL have a higher theoretical sweetness than a dough with 112.5 mgEP/kg flour of AMG Goldcrust® and a dough with 53.6 mgEP/kg flour AMG NL generates more glucose than a dough with 112.5 mgEP/kg flour of AMG Goldcrust®.

TABLE 31

| | Dough | | | | | | | |
|---|---|---|---|---|---|---|---|---|
| | 1 | 2 | 3 | 4 | 5 | 6 | 7 | 8 |
| AMG | 112.5 | — | — | — | — | — | — | — |
| Goldcrust ®, | | | | | | | | |
| mgEP/kg | | | | | | | | |
| AMG NL, | | 17.9 | 26.8 | 35.7 | 44.6 | 53.6 | 62.5 | 71.4 |
| mgEP/kg | | | | | | | | |
| Glucose, mg/g | 17.6 | 11.4 | 13.5 | 15.3 | 17.0 | 18.9 | 19.7 | 21.4 |
| Fructose, mg/g | 10.7 | 10.3 | 10.7 | 10.8 | 10.6 | 10.7 | 10.7 | 10.7 |
| Maltose, mg/g | 12.1 | 16.1 | 16.0 | 16.0 | 15.6 | 15.7 | 14.2 | 13.8 |
| Maltotriose, | 0.3 | 0.5 | 0.5 | 0.5 | 0.4 | 0.4 | 0.4 | 0.4 |
| mg/g | | | | | | | | |
| Sweetness | 21.9 | 19.3 | 20.7 | 21.6 | 22.2 | 23.3 | 23.4 | 24.2 |
| calculation* | | | | | | | | |

### Example 11. Dosage Response of AMG anPAV498 (Partial Sugar Replacement)

Bread was baked in a straight dough process with a recipe according to Table 32. The ingredients were mixed in a spiral mixer into a dough for 3+7 min at 17 respectively 35 rpm. The doughs were divided into 450 g pieces, rounded, sheeted and place in baking tins. The tins with the doughs were proofed for 55 min at 32° C. and 86% relative humidity. The proofed doughs were baked in a deck oven for 25 min at 230° C.

TABLE 32

| | Bakers % |
|---|---|
| Flour (Kolibri, Meneba, NL) | 100 |
| Water | 55.5 |
| Yeast | 3 |
| Sucrose | 5 |
| Salt | 0.5 |
| Ascorbic acid | 0.04 |
| Calcium Propionate | 0.25 |
| Fungamyl 4000SG | 7 ppm |
| Panzea BG | 25 ppm |

TABLE 33
Eight treatments were prepared with different enzymatic additions

| | Dough | | | | | | | |
|---|---|---|---|---|---|---|---|---|
| | 1 | 2 | 3 | 4 | 5 | 6 | 7 | 8 |
| Goldcrust ®, | 112.5 | — | — | — | — | — | — | — |
| mgEP/kg | | | | | | | | |
| AMG | — | 12.0 | 15.1 | 18.1 | 21.1 | 24.1 | 27.1 | 30.1 |
| anPAV498 | | | | | | | | |
| mgEP/kg | | | | | | | | |
| Ja126 | 0.35 | 0.35 | 0.35 | 0.35 | 0.35 | 0.35 | 0.35 | 0.35 |
| mgEP/kg | | | | | | | | |

The dough properties were evaluated by a trained baker and the volume of the bread was determined using Volscan profiler (Stable microsystems, Godalming, UK). The results from the evaluation can be found in

Table 34, All the doughs had similar dough properties and produced bread with similar volume.

TABLE 34

| | Dough | | | | | | | |
|---|---|---|---|---|---|---|---|---|
| | 1 | 2 | 3 | 4 | 5 | 6 | 7 | 8 |
| Stickiness | 5 | 5 | 5 | 5 | 5 | 5 | 5 | 5 |
| Softness | 5 | 5 | 5 | 5 | 6 | 6 | 6 | 6 |
| Extensibility | 5 | 5 | 5 | 6 | 6 | 6 | 6 | 6 |
| Elasticity | 5 | 6 | 6 | 5 | 5 | 5 | 5 | 5 |
| Average spec | 4.3 | 4.4 | 4.3 | 4.3 | 4.3 | 4.2 | 4.2 | 4.2 |
| vol ml/g | | | | | | | | |
| Spec vol | 100 | 101 | 100 | 99 | 100 | 97 | 98 | 96 |
| index % | | | | | | | | |

Sugars were extracted from the bread crumb using 0.1 M Phosphate buffer pH 8.0 in 70% EtOH. Bread crumb (180 mg) were added to the extraction buffer (1.8 ml) and was incubated for 20 minutes at 70° C. during mixing. The bread crumb was spun down at 12 000 rpm for 5 minutes in a centrifuge and 500 μl of the supernatant was taken and diluted 200× using a 20 mM Phosphate buffer pH 8.0+10 mg/L cellobiose as internal standard. The extracted sugars (glucose, fructose, maltose and maltotriose) were quantified on an ICS-5000 HPLC system with a CarboPac PA1 column. A theoretical sweetness was calculated based on the levels of glucose, fructose and maltose was calculated using sweetness intensity factors. The sweetness factors in Table 35 were based on the determinations in Portmann M O, Birch G. J Sci Food Agric 69(3):275-81, 1995.

TABLE 35

| Sugar | Sweetness intensity factor |
|---|---|
| Glucose | 0.5 |
| Fructose | 1 |
| Maltose | 0.2 |

The amount of sugars (g/kg bread crumb) and is theoretical sweetness can be found in Table 36. Based on these sugar levels it can be calculated that a dough with 24.1 mgEP/kg flour of AMG anPAV498 generates a higher theoretical sweetness than a dough with 112.5 mgEP/kg flour of Goldcrust® and a dough with 27.1 mgEP/kg flour AMG anPAV498 generates more glucose than a dough with 112.5 mgPE/kg flour of Goldcrust®.

TABLE 36

| | Dough | | | | | | | |
|---|---|---|---|---|---|---|---|---|
| | 1 | 2 | 3 | 4 | 5 | 6 | 7 | 8 |
| Glucose, mg/g | 18.0 | 12.1 | 14.2 | 14.8 | 16.2 | 17.3 | 18.4 | 19.0 |
| Fructore, mg/g | 10.6 | 10.2 | 10.6 | 10.5 | 10.4 | 10.4 | 10.3 | 10.2 |
| Maltose, mg/g | 11.3 | 16.3 | 16.9 | 16.5 | 16.3 | 16.1 | 15.8 | 15.3 |
| Maltotriose, | 0.3 | 0.6 | 0.7 | 0.6 | 0.6 | 0.6 | 0.5 | 0.5 |
| mg/g | | | | | | | | |
| Sweetness | 21.9 | 19.5 | 21.1 | 21.2 | 21.8 | 22.3 | 22.7 | 22.7 |
| calculation* | | | | | | | | |

### Example 12. Sensory Comparison of Sweetness of Goldcrust® to AMG NL and AMG anPAV498 (Partial Sugar Replacement)

Bread was baked in a straight dough process with a recipe according to Table 37. The ingredients were mixed in a spiral mixer into a dough for 3+8 min at 17 respectively 35 rpm. The doughs were divided into 350 g pieces, rounded, sheeted and place in baking tins. The tins with the doughs were proofed for 85 and 115 min at 35° C. and 85% relative humidity. The proofed doughs were baked in a deck oven for 25 min at 230° C.

TABLE 37

| | Bakers % |
|---|---|
| Flour | 100 |
| Water | 57 |
| Fresh Yeast | 3 |
| Salt | 1 |
| Sugar | 5 |
| Ascorbic acid | 0.06 |
| Fungamyl 4000SG | 7 ppm |
| Panzea BG | 25 ppm |

TABLE 38
Three treatments were prepared with different enzymatic additions

| Dough | 1 | 2 | 3 |
|---|---|---|---|
| Goldcrust ®, mgEP/kg | 124.3 | | |
| AMG NL, mgEP/kg | | 52.2 | |
| AMG anPAV498, mgEP/kg | | | 23.6 |
| Ja126 mgEP/kg | 0.34 | 0.34 | 0.34 |

TABLE 39
Dough properties

| Dough | 1 | 2 | 3 |
|---|---|---|---|
| Average spec vol ml/g | 7.1 | 7.0 | 7.2 |
| Spec vol index % | 100 | 99 | 102 |

Sugars were extracted from the bread crumb using 0.1 M Phosphate buffer pH 8.0 in 70% EtOH. Bread crumb (180 mg) were added to the extraction buffer (1.8 ml) and was incubated for 20 minutes at 70° C. during mixing. The bread crumb was spun down at 12 000 rpm for 5 minutes in a centrifuge and 500 μl of the supernatant was taken and diluted 200× using a 20 mM Phosphate buffer pH 8.0+10 mg/L cellobiose as internal standard. The extracted sugars (glucose, fructose, maltose and maltotriose) were quantified on an ICS-5000 HPLC system with a CarboPac PA1 column. A theoretical sweetness was calculated based on the levels of glucose, fructose and maltose was calculated using sweetness intensity factors. The sweetness factors in Table 40 were based on the determinations in Portmann M O, Birch G. J Sci Food Agric 69(3):275-81, 1995.

TABLE 40

| Sugar | Sweetness intensity factor |
|---|---|
| Glucose | 0.5 |
| Fructose | 1 |
| Maltose | 0.2 |

The amount of sugars (mg/g bread crumb) and its theoretical sweetness can be found in Table 41 Table 316. AMG Goldcrust® generates the more glucose, while the level of maltose is higher for AMG NL and AMG anPAV498. However, the calculated sweetness of AMG NL at 52.2 mgEP/kg flour and AMG anPAV498 at 23.6 mgEP/kg flour is actually similar to that of Goldcrust® at a far higher dosing of 124.3 mgEP/kg flour (table 32).

TABLE 41

| Dough | 1 | 2 | 3 |
|---|---|---|---|
| Glucose, mg/g | 18.9 | 17.7 | 15.9 |
| Fructose, mg/g | 8.8 | 8.3 | 7.9 |
| Maltose, mg/g | 4.8 | 8.6 | 11.5 |
| Maltotriose, mg/g | M | M | M |
| Sweetness calculation* | 19.3 | 18.9 | 18.2 |

### Sensory Evaluation Method

Each sensory assessor was served 2 slices of each bread type (day 1). Samples were served blind, 3-digit coded, and in random order. 7 assessors participated in the evaluation. Intensity of the bread crumb sweet taste was evaluated on a 1-9 point intensity scale ranging from little to very intense.

The sweetness did not differ significantly between samples and no other significant differences were noted between samples.

TABLE 42

| Dough | 1 | 2 | 3 |
|---|---|---|---|
| Sweet taste | 5.6 | 6.0 | 5.6 |

### Example 13. Sensory Evaluation, Full Sugar Replacement

Toast bread (panned bread, open top) —no sucrose added to doughs

TABLE 43
Recipe, % (w/w):

| Ingredients: | Baker's % |
|---|---|
| Wheat flour, Kolibri % | 100 |
| Fresh yeast % | 4 |
| Salt % | 0.5 |
| Water % | 57.5 |
| Enzyme solution | * |
| Fungamyl ® 4000 SG ppm | 7 |
| Panzea ® BG ppm | 25 |
| Ascorbic acid ppm | 40 |

* Enzyme solutions:
Control (=no starch degrading enzyme and no glucoamylase)
Enzyme solution A: 0.35 mg raw starch degrading alpha-amylase (JA126) protein per kg flour and 112.5 mg Goldcrust ® glucoamylase protein per kg flour
Enzyme solution B: 0.35 mg raw starch degrading alpha-amylase (JA126) protein per kg flour, and 53.6 mg AMG NL glucoamylase protein per kg flour
Enzyme solution C: 0.35 mg raw starch degrading alpha-amylase (JA126) protein per kg flour, and 21.1 mg AMG anPAV498 glucoamylase protein per kg flour

TABLE 44
Baking procedure

| Procedure | Time, min |
|---|---|
| Mixing at speed low/high speed (17 rpm/35 rpm) | 3/7 |
| Temperature after mixing, ° C. | 25.3-26.2 |
| Floor time | 20 |
| Scaling 320 g, in baking tins | 10 |
| Table resting/bench time | 15 |
| Fermentation time at 32° C., min | 80 |
| Baking at temperature 230° C. | 25 |

### Sensory Evaluation Method

Each assessor was served 2 slices of each bread type (day 1). Samples were served blind, 3-digit coded, and in random order. Moist and Soft were evaluated by hand, and sweet by tasting the breadcrumb. The sensory attributes were evaluated on a 1-9 point intensity scale, ranging from little to very. 4 trained assessors participated in the evaluation. Two sensory replicates were performed.

### Results:

The doughs had same stickiness and softness. AMG-NL gave more extensible and less elastic dough (Table 45).

TABLE 45
Dough parameters

| | Control | A | B | C |
|---|---|---|---|---|
| Stickiness | 5 | 6 | 6 | 6 |
| Softness | 5 | 5 | 5 | 5 |
| Extensibility | 5 | 5 | 6 | 5 |
| Elasticity | 5 | 5 | 4 | 5 |

The data shown in Table 45 demonstrate that solution C gave most moist and soft bread, whereas for sweet taste there was no significant difference No other differences were noted between samples. There was no significant difference in bread specific volume (Table 46).

TABLE 46
Specific volume index, %

| Control | A | B | C |
|---|---|---|---|
| 100 | 101 | 102 | 103 |

TABLE 47
Mean sensory scores of the enzyme bread, 1 day after baking.

| Sensory attribute | A | B | C | p-value |
|---|---|---|---|---|
| Moist | 5.1AB | 5.0B | 5.8B | 0.0338 |
| Soft | 5.1B | 5.1B | 6.6A | 0.002 |
| Sweet taste | 4.0 | 3.6 | 4.0 | 0.7998 |

Tukey HSD: Means followed by different letters within sensory attribute were significantly (P < 0.05) different between samples

Sugars were extracted from the bread crumb using 0.1 M Phosphate buffer pH 8.0 in 70% EtOH. Bread crumb (180 mg) were added to the extraction buffer (1.8 ml) and was incubated for 20 minutes at 70° C. during mixing. The bread crumb was spun down at 12 000 rpm for 5 minutes in a centrifuge and 500 μl of the supernatant was taken and diluted 200× using a 20 mM Phosphate buffer pH 8.0+10 mg/L cellobiose as internal standard. The extracted sugars (glucose, fructose, maltose and maltotriose) were quantified on an ICS-5000 HPLC system with a CarboPac PA1 column.

The glucose levels in the breads were slightly higher with B and C than A (Table 48), which means improved sweetness for B and C compared to the control A. Maltose was higher with B and C than with A.

TABLE 48
Sugar levels (mg/g bread crumb) in the bread

| | Glucose mg/g | Fructose mg/g | Maltose mg/g |
|---|---|---|---|
| Enzyme solution | bread | bread | bread |
| Control | 0.5 | 0.1 | 11.2 |
| A | 9.6 | 1.4 | 6.6 |
| B | 10.9 | 0.3 | 10.6 |
| C | 11.3 | 0.1 | 10.7 |

### Example 14. Freshness Effect of AMGs in US Sponge and Dough Recipe

Bread was baked in a sponge and dough process with a recipe according to Table 49. The bread was baked in lidded tins in order to have the same volume of all bread. The ingredients of the sponge were mixed in a pin mixer into a dough for 2+1 min at 50 respectively 150 rpm. The sponge was proofed for 2 hours at 27° C. and 75% rH. The sponge was placed in the pin mixer with the rest of the ingredients of the dough and mixed into a dough for 1+3 minutes at 50 and 150 rpm respectively.

The doughs were divided into 400 gram pieces, rounded, sheeted and place in baking tins with lid. The tins with the doughs were proofed for 60 min at 43° C. and 80% relative humidity. The proofed doughs were baked in a revolving oven for 20 min at 215° C.

TABLE 49
Recipe

| | |
|---|---|
| Ingredient | Amount |

Sponge

| | |
|---|---|
| Flour, % | 70 |
| Water, % | 40.6 |
| Dry yeast, % | 2.3 |
| SSL, % | 0.5 |
| Soybean oil, % | 3 |
| Dimodan HP75, % | 0.5 |
| Calcium propionate, % | 0.1 |

Dough

| | |
|---|---|
| Flour, % | 30 |
| Water, % | 16.4 |
| Sugar, % | 8 |
| Salt, % | 2.3 |
| Calcium Propionate, % | 0.4 |
| Ascorbic acid, ppm | 60 |
| Fungamyl ® 4000 SG, ppm | 6 |
| Panzea ® BG, ppm | 25 |

TABLE 50
Seven treatments were prepared with different enzymatic additions

| | | | | | | |
|---|---|---|---|---|---|---|
| 1 | 2 | 3 | 4 | 5 | 6 | 7 |

| | | | | | | |
|---|---|---|---|---|---|---|
| AMG anPAV498, | | | 25 | 50 | | |
| mgEP/kg flour | | | | | | |
| JPO124, | | 25 | | | 50 | |
| mgEP/kg flour | | | | | | |
| JPO172, | 25 | | | | | 50 |
| mgEP/kg flour | | | | | | |

The bread was packed 2 hours after baking in sealed plastic bags and stored at room temperature until analysis. The texture of the bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product. A standard method for measuring firmness and elasticity is based on force-deformation of the baked product. A force-deformation of the baked products may be performed with a 40 mm diameter cylindrical probe. The force on the cylindrical probe is recorded as it is pressed down 40% strain on a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 40% strain (corresponding to force at time=40 s for a bread slice of 25 mm thickness) divided by the force needed to press the probe 10 mm into the crumb (corresponding to force at time=10 s for a bread slice of 25 mm thickness) times 100. The results from the texture evaluation can be found in Tables 46 and 47, respectively.

The control bread increased in firmness (table 51) and lost elasticity (table 52) over storage time, also known as bread staling.

Surprisingly, all three AMGs tested in this study had an anti-staling effect seen as less increase in firmness over storage time—table 46. The AMGs also had a positive effect on elasticity that starts at a higher level, and after 14 days of storage the AMG breads had a higher elasticity than the control—table 47.

TABLE 51
Firmness over storage time

| Treatment | Day 1 | Day 7 | Day 14 |
|---|---|---|---|
| Control | 168 | 490 | 750 |
| AMG anPAV498, 25 mgEP/kg flour | 191 | 423 | 667 |
| AMG anPAV498, 50 mgEP/kg flour | 182 | 387 | 561 |
| JPO124, 25 mgEP/kg flour | 146 | 374 | 572 |
| JPO124, 50 mgEP/kg flour | 143 | 319 | 465 |
| JPO172, 25 mgEP/kg flour | 124 | 318 | 447 |
| JPO172, 50 mgEP/kg flour | 143 | 301 | 434 |

TABLE 52
Elasticity over storage time

| Treatment | Day 1 | Day 7 | Day 14 |
|---|---|---|---|
| Control | 52.8 | 49.9 | 44.7 |
| anPAV498, 25 mgEP/kg flour | 53.9 | 50.1 | 46.8 |
| anPAV498, 50 mgEP/kg flour | 55.9 | 51.6 | 48.1 |
| JPO124, 25 mgEP/kg flour | 55.7 | 52.2 | 48.4 |
| JPO124, 50 mgEP/kg flour | 58.0 | 54.1 | 51.7 |
| JPO172, 25 mgEP/kg flour | 55.6 | 53.3 | 50.2 |
| JPO172, 50 mgEP/kg flour | 59.8 | 56.3 | 52.7 |

### Example 15. Use of Thermostable AMG in Cake Applications

Muffins were baked using a commercial cake mix (Tegral Satin Creame Cake Neutral SG, Puratos, UK) using the recipe on the bag.

TABLE 53
Muffin recipe.

| Ingredient | Amount, % |
|---|---|
| Tegral satin cream cake mix | 100 |
| Egg | 40 |
| Oil (rapeseed oil) | 30 |
| Water | 22.5 |

TABLE 54
Nine muffin treatments were prepared
with different enzyme additions:

1. Blank
2. JPO124 100 mgEP/kg cake mix
3. JPO124 900 mgEP/kg cake mix
4. JPO172 100 mgEP/kg cake mix
5. JPO172 900 mgEP/kg cake mix
6. OC50 1250 MANU/kg cake mix (6.25 mgEP/kg cake mix)
7. OC50 2500 MANU/kg cake mix (12.5 mgEP/kg cake mix)
8. OC50 3750 MANU/kg cake mix (18.75 mgEP/kg cake mix)
9. OC50 5000 MANU/kg cake mix (25 mgEP/kg cake mix)

TABLE 55
Muffin making process:

1. Eggs, oil and water according to table 48 were added
to the mixing bowl.
2. The individual treatments, according to table 49,
were added to each dough
3. The cakemix was added to the mixing bowl and mixed for
1 min with a hand mixer on
speed 1 into a cake batter.
4. The cake batter was placed in muffin tin (50 g batter
to each tin) using a piping bag.
5. The muffins were baked for 28 minutes in a deck oven
with a top heat at 200° C. and bottom heat at 180° C.
and with a tray upside down in the bottom of the oven.
6. The muffins were allowed to cool down for 1 h and
placed in sealed plastic bag with modified
atmosphere and stored at room temperature until analysis.

The textural properties of the muffin were analyzed using a texture analyzer performing a texture profile analysis (TPA). In the analysis of the muffin, the top of the muffin was cut off at the same level as the muffin tin leaving a 3 cm muffin. The muffin was placed on the texture analyzer and a 25 mm diameter cylindrical probe was pressed down into the muffin twice to a 7 mm depth at a constant upward and downward speed of 1 mm/s with 5 seconds between the two compressions. The force (gram) as a function of time (seconds) and distance (mm) was recorded.
The peak force of the first compression corresponds to the hardness (gram) of the muffin.
The area below the force-distance curve of the second compression divided by the area below the force-distance curve of the first compression corresponds to cohesiveness and were expressed in %.
The area below the force-distance curve of the first upward move divided by the area below the first downward move corresponds to resilience and were expressed in %.
   Table 56 below illustrates the benefits of using JPO172 and JPO124 in muffins. The muffins treated with JPO124 and JPO172 have a surprisingly improved (higher) resilience and Cohesiveness; even higher than other known solutions for improving cake freshness.

TABLE 56
Textural properties of muffins with different enzymatic treatments.

| Treatment | Hardness | Resilience, % | Cohesiveness, % |
|---|---|---|---|
| Blank | 174 | 30.2 | 66.4 |
| JPO124 100 me/ | 175 | 30.2 | 66.4 |
| kg cake mix | | | |
| JPO124 900 mgEP/ | 184 | 31.5 | 67.2 |
| kg cake mix | | | |
| JPO172 100 mgEP/ | 174 | 32.8 | 68.7 |
| kg cake mix | | | |
| JPO172 900 mgEP/ | 177 | 33.7 | 69.7 |
| kg cake mix | | | |
| OC50 1250 MANU/ | 160 | 30.3 | 66.9 |
| kg cake mix | | | |
| (6.25 mgEP) | | | |
| OC50 2500 MANU/ | 159 | 30.7 | 67.3 |
| kg cake mix | | | |
| (12.5 mgEP) | | | |
| OC50 3750 MANU/ | 157 | 31.6 | 68.2 |
| kg cake mix | | | |
| (18.75 mgEP) | | | |
| OC50 5000 MANU/ | 155 | 31.9 | 68.1 |
| kg cake mix | | | |
| (25 mgEP) | | | |

### Example 16. Sensory Comparison of Freshness of AMG Goldcrust®, AMG NL, AMG AnPAV498, JP172 and Novamyl 3D®

Bread was baked in a straight dough process with a recipe according to Table 57. The ingredients were mixed in a spiral mixer into a dough for 3+6 min at 17 respectively 35 rpm. The doughs were divided into 450 g pieces, rounded, sheeted and placed in baking tins. The tins with the doughs were proofed for 55 mins at 32° C. and 86% relative humidity. The proofed doughs were baked in a deck oven for 35 min at 230° C.

TABLE 57

| | Bakers % |
|---|---|
| Flour | 100 |
| Water | 57 |
| Fresh Yeast | 4.5 |
| Salt | 1.5 |
| Sugar | 1.5 |
| Calcium propionate | 0.25 |
| Ascorbic acid | 0.04 |
| Fungamyl 4000SG | 7 ppm |
| Panzea BG | 25 ppm |

TABLE 58
Treatments were prepared with different enzymatic additions

Dough

| | | | | | |
|---|---|---|---|---|---|
| 1 | 2 | 3 | 4 | 5 | 6 |

| | | | | | |
|---|---|---|---|---|---|
| Control (no | | | | | |
| glucoamylase) | | | | | |
| Goldcrust ® | 50 | | | | |
| 3300 BG, | | | | | |
| mgEP/kg | | | | | |
| AMG NL | | 50 | | | |
| mgEP/kg | | | | | |
| AMG | | | 500 | | |
| AnPAV498 | | | | | |
| mgEP/kg*⁾ | | | | | |
| JPO172 | | | | 50 | |
| mgEP/kg | | | | | |
| Novamyl | | | | | 25 |
| 3D ppm | | | | | |

*⁾Note:
AMG AnPAV498 was mistakenly overdosed ten times in this experiment; it should have been 50 mgEP/kg, but was 500 mgEP/kg.

### Sensory Evaluation Method

Sensory evaluation was performed on day 1 and day 8. A training session was held prior to evaluation, identifying the relevant attributes and procedures (Table 59). Texture was evaluated by hand. 4-5 trained assessors participated in the evaluation. Each assessor was served 2 slices without crust of each bread type. Samples were served blind, 3-digit coded, and in random order. Intensity of the sensory attributes were evaluated on a 1-9 point intensity scale ranging from little to very intense. Two sensory replicates were performed on each evaluation day.

TABLE 59
Description of sensory attributes, procedures and evaluation

| Attribute | Procedure | Evaluate |
|---|---|---|
| Moist | Touch the surface of the | Degree of moistness/ |
| | bread, hold for 3 sec | cooling perceived |
| Soft | Gently compress the bread | Ease of compressing the crumb |
| Resilient/ | Compress the slice | Degree to which the crumb |
| springy | completely | recovers to original shape |

Use the other bread slice

| | | |
|---|---|---|
| Foldable | Fold the bread slice | Extent of coherency at |
| | | the fold |

### Sensory Results

JPO172 and AMG AnPAV498 scored highest on all the evaluated freshness attributes on day 1, and on Moist, Soft and foldable Day 8. AMG Goldcrust® did not differ from Control.

TABLE 60
Mean values of sensory scores of the bread day 1.

| Day 1 | Moist | Soft | Resilient/Springy | Foldable |
|---|---|---|---|---|
| Control | 5.8 | 5.6 | 6.3 | 5.5 |
| 50 mgEP/kg | 5.8 | 5.1 | 6.6 | 6.0 |
| Goldcrust ® | | | | |
| 50 mgEP/kg | 8.0 | 8.0 | 7.6 | 9.0 |
| JPO172 | | | | |
| 50 mgEP/kg | 6.1 | 6.1 | 6.6 | 6.7 |
| AMG NL | | | | |
| 500 mgEP/kg | 8.2 | 8.1 | 7.9 | 8.8 |
| AMG AnPAV498 | | | | |
| 25 ppm Novamyl | 5.8 | 6.4 | 6.0 | 7.1 |
| 3D | | | | |

TABLE 61
Mean values of sensory scores of the bread day 8.

| Day 8 | Moist | Soft | Resilient/Springy | Foldable |
|---|---|---|---|---|
| Control | 2.3 | 2.0 | 7.3 | 1.2 |
| 50 mgEP/kg | 2.4 | 1.7 | 7.7 | 1.7 |
| Goldcrust ® | | | | |
| 50 mgEP/kg | 6.1 | 6.5 | 7.8 | 7.5 |
| JPO172 | | | | |
| 50 mgEP/kg flour | 2.5 | 2.6 | 7.7 | 1.8 |
| AMG NL | | | | |
| 500 mgEP/kg | 6.3 | 6.9 | 7.8 | 8.1 |
| AMG AnPAV498 | | | | |
| 25 ppm Novamyl | 4.2 | 4.7 | 6.6 | 4.6 |
| 3D | | | | |

### Example 17. Freshness Effect of AMG the First 24 Hours

Bread was baked in a straight dough mini baking process with a recipe according to

Table 62. The bread was baked in lidded tins in order to have the same volume of all bread. The ingredients were mixed in a spiral mixer into a dough for 4 minutes at 90 rpm. The doughs were divided into 20 g pieces, rounded and place in baking tins. The tins with the doughs were proofed on a conveyor belt for 55 min at 36° C. and 80% relative humidity. The proofed doughs were baked in mini tunnel oven for 12 min at 210° C.

TABLE 62

| | Bakers % |
|---|---|
| Flour (Kolibri, Meneba, NL) | 100 |
| Water | 58 |
| Yeast | 4.5 |
| Sucrose | 1.5 |
| Salt | 1.5 |
| Ascorbic acid | 0.04 |
| Calcium Propionate | 0.3 |
| Fungamyl ® 4000SG | 8 ppm |
| Panzea ® BG | 25 ppm |

TABLE 63
Ten dough treatments were prepared
with different enzymatic additions

1. Control (Blank)
2. Datem 0.5%
3. JPO172 50 mgEP/kg flour
4. Opticake 50 BG 200 MANU/kg
5. JPO124 50 mgEP/kg flour
6. Novmayl 3D 440 MANU/kg flour
7. SSL 0.5%
8. Distilled monoglycerides 0.5%
9. Novamyl 10 000 BG 750 MANU/kg
10. Lipopan Extra 200 LU/kg

The doughs were baked and the resulting breads were packed 0.5 hours after baking in sealed plastic bags and stored at room temperature until analysis.

The texture of each bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product.

A standard method for measuring firmness and elasticity is based on force-deformation of the baked product. A force-deformation of the baked products may be performed with a 20 mm diameter spherical probe. The force on the probe is recorded as it is pressed down 40% strain on a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 40% strain (corresponding to force at time=40 s for a bread slice of 25 mm thickness) divided by the force needed to press the probe 10 mm into the crumb (corresponding to force at time=10 s for a bread slice of 25 mm thickness) times 100.

The results from the texture analysis can be found in Table 64 (firmness) and Table 65 (elasticity).

Fresh bread without enzyme (control) has low firmness and high elasticity, as the bread is stored the firmness increase over time and the elasticity decrease. Traditional AMGs used in baking applications (for example AMG Goldcrust®) do not impact the firmness or elasticity (see Example 7).

Two of the AMGs herein (JPO124 and JPO172) dosed at 50 mgEP/kg improved (reduced) the increase in firmness over time. Both AMGs (JPO124 and JPO172) dosed at 50 mgEP/kg improved (increased) the initial elasticity and prevented the loss of elasticity over time.

TABLE 64
Firmness (g) 2, 5 and 24 hours after baking of bread
with enzyme treatments according Table 63.

| Treatment | 2 Hour | 5 hours | 24 hour |
|---|---|---|---|
| Control | 166 | 189 | 463 |
| JPO124 50 mgEP/kg | 165 | 175 | 311 |
| JPO172 50 mgEP/kg | 163 | 166 | 236 |
| Novamyl 10 000 BG | 166 | 188 | 358 |
| 750 MANU/kg | | | |
| Novamyl 3D 440 MANU/kg | 155 | 181 | 329 |
| OC50 200 MANU/kg | 175 | 180 | 325 |
| Lipopan Xtra 200 LU/kg | 145 | 156 | 334 |
| DMG 0.5% | 188 | 240 | 482 |
| DATEM 0.5% | 163 | 173 | 403 |
| SSL 0.5% | 194 | 193 | 407 |

TABLE 65
Elasticity (%) 2, 5 and 24 hours after baking of
bread with enzyme treatments according Table 63.

| Treatment | 2 hours | 5 hours | 24 hours |
|---|---|---|---|
| Control | 64.5 | 63.8 | 55.9 |
| JPO124 50 mgEP/kg | 66.2 | 66.6 | 63.2 |
| JPO172 50 mgEP/kg | 66.2 | 66.7 | 65.2 |
| Novamyl 10 000 BG | 61.8 | 61.5 | 59.0 |
| 750 MANU/kg | | | |
| Novamyl 3D 440 MANU/kg | 63.3 | 61.9 | 58.1 |
| OC50 200 MANU/kg | 62.2 | 63.5 | 59.4 |
| Lipopan Xtra 200 LU/kg | 62.0 | 61.4 | 54.6 |
| DMG 0.5% | 62.2 | 61.2 | 54.8 |
| DATEM 0.5% | 63.4 | 62.9 | 56.3 |
| SSL 0.5% | 58.2 | 57.8 | 51.8 |

### Example 18. Freshness Effect of AMG at High Dosages

Bread was baked in a straight dough mini baking process with a recipe according to

Table 66. The bread was baked in lidded tins in order to have the same volume of all bread. The ingredients were mixed in a spiral mixer into a dough for 4 minutes at 90 rpm. The doughs were divided into 20 g pieces, rounded and place in baking tins. The tins with the doughs were proofed on a conveyor belt for 55 min at 36° C. and 80% relative humidity. The proofed doughs were baked in mini tunnel oven for 12 min at 210° C.

TABLE 66

| | Bakers % |
|---|---|
| Flour (Kolibri, Meneba, NL) | 100 |
| Water | 58 |
| Yeast | 4.5 |
| Sucrose | 1.5 |
| Salt | 1.5 |
| Ascorbic acid | 0.04 |
| Calcium Propionate | 0.3 |
| Fungamyl ® 4000SG | 8 ppm |
| Panzea ® BG | 25 ppm |

TABLE 67
Ten treatments were prepared with different enzymatic additions

1. Control
2. Opticake 50BG 200 MANU/kg
3. JPO124 50 mgEP/kg flour
4. JPO124 100 mgEP/kg flour
5. JPO124 300 mgEP/kg flour
6. JPO124 500 mgEP/kg flour
7. JPO172 50 mgEP/kg flour
8. JPO172 100 mgEP/kg flour
9. JPO172 300 mgEP/kg flour
10. JPO172 500mgEP/kg flour

The doughs were baked and the resulting breads were packed 0.5 hours after baking in sealed plastic bags and stored at room temperature until analysis.

The texture of each bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product.

A standard method for measuring firmness and elasticity is based on force-deformation of the baked product. A force-deformation of the baked products may be performed with a 20 mm diameter spherical probe. The force on the probe is recorded as it is pressed down 40% strain on a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 40% strain (corresponding to force at time=40 s for a bread slice of 25 mm thickness) divided by the force needed to press the probe 10 mm into the crumb (corresponding to force at time=10 s for a bread slice of 25 mm thickness) times 100.

The results from the texture analysis can be found in Table 68 (firmness) and Table 69 (elasticity).

Fresh bread without enzyme (control) has low firmness and high elasticity, as the bread is stored the firmness increase over time and the elasticity decrease. Traditional AMGs used in baking applications (for example AMG Goldcrust®) do not impact the firmness or elasticity (see Example 7).

The two new AMGs (JPO124 and JPO172) improved (reduced) initial firmness and the increase in firmness over time. The higher dosage the lower increase in firmness over time. Both AMGs (JPO124 and JPO172) improved (increased) the initial elasticity and prevented the loss of elasticity over time. The higher dosage of the AMG the higher initial elasticity and the lower loss of elasticity over time.

TABLE 68
Firmness (g) on day 1 and 7 of bread with
enzyme treatments according Table 67.

| Treatment | Day 1 | Day 7 |
|---|---|---|
| Control | 259 | 1083 |
| Opticake 50BG 200 MANU/kg flour | 217 | 451 |
| JPO124 50 mgEP/kg flour | 185 | 639 |
| JPO124 100 mgEP/kg flour | 159 | 416 |
| JPO124 300 mgEP/kg flour | 146 | 230 |
| JPO124 500 mgEP/kg flour | 201 | 237 |
| JPO172 50 mgEP/kg flour | 174 | 463 |
| JPO172 100 mgEP/kg flour | 145 | 369 |
| JPO172 300 mgEP/kg flour | 162 | 210 |
| JPO172 500 mgEP/kg flour | 209 | 280 |

TABLE 69
Elasticity (%) on day 1 and 7 of bread with
enzyme treatments according Table 67.

| Treatment | Day 1 | Day 7 |
|---|---|---|
| ontrol | 57.2 | 45.3 |
| Opticake 50BG 200 MANU/kg flour | 60.2 | 52.9 |
| JPO124 50 mgEP/kg flour | 63.9 | 53.1 |
| JPO124 100 mgEP/kg flour | 65.9 | 59.2 |
| JPO124 300 mgEP/kg flour | 68.4 | 67.3 |
| JPO124 500 mgEP/kg flour | 69.0 | 68.7 |
| JPO172 50 mgEP/kg flour | 64.5 | 57.1 |
| JPO172 100 mgEP/kg flour | 66.3 | 62.5 |
| JPO172 300 mgEP/kg flour | 67.6 | 67.2 |
| JPO172 500 mgEP/kg flour | 68.4 | 68.6 |

### Example 19. Freshness Effect of AMG Combined with Lip182

Bread was baked in a straight dough mini baking process with a recipe according to

Table 10. The bread was baked in lidded tins in order to have the same volume of all bread. The ingredients were mixed in a spiral mixer into a dough for 4 minutes at 90 rpm. The doughs were divided into 20 g pieces, rounded and place in baking tins. The tins with the doughs were proofed on a conveyor belt for 55 min at 36° C. and 80% relative humidity. The proofed doughs were baked in mini tunnel oven for 12 min at 210° C.

TABLE 70

| | Bakers % |
|---|---|
| Flour (Kolibri, Meneba, NL) | 100 |
| Water | 58 |
| Yeast | 4.5 |
| Sucrose | 1.5 |
| Salt | 1.5 |
| Ascorbic acid | 0.04 |
| Calcium Propionate | 0.3 |
| Fungamyl ® 4000SG | 8 ppm |
| Panzea ® BG | 25 ppm |

TABLE 71
Various treatments were prepared with
different enzymatic additions

| | Opticake 50BG, | JPO172, | Lip182, |
|---|---|---|---|
| | MANU/kg | mgEP/kg | PLA(B)/kg |
| Treatment | flour | flour | flour |
| 1 | | | |
| 2 | 200 | | |
| 3 | | | 2.5 |
| 4 | | | 5 |
| 5 | | 25 | |
| 6 | | 50 | |
| 7 | | 25 | 2.5 |
| 8 | | 25 | 5 |
| 9 | | 50 | 2.5 |
| 10 | | 50 | 5 |

The doughs were baked and the resulting breads were packed 0.5 hours after baking in sealed plastic bags and stored at room temperature until analysis.

The texture of each bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product.

A standard method for measuring firmness and elasticity is based on force-deformation of the baked product. A force-deformation of the baked products may be performed with a 20 mm diameter spherical probe. The force on the probe is recorded as it is pressed down 40% strain on a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 40% strain (corresponding to force at time=40 s for a bread slice of 25 mm thickness) divided by the force needed to press the probe 10 mm into the crumb (corresponding to force at time=10 s for a bread slice of 25 mm thickness) times 100.

The results from the texture analysis can be found in Table 72 (firmness) and Table 73 (elasticity).

Fresh bread without enzyme (control) has low firmness and high elasticity, as the bread is stored the firmness increase over time and the elasticity decrease. Traditional AMGs used in baking applications (for example, AMG Goldcrust®) does not impact the firmness or elasticity (see Example 7).

The AMG JPO172 improved (reduced) initial firmness and the increase in firmness over time. The lipase Lip182 had no effect on firmness alone compared to a control bread. The combination of Lip182 and JPO172 resulted in the bread with lowest firmness on both day 1 and 7.

The AMGs JPO172 improved (increased) the initial elasticity and prevented the loss of elasticity over time. The lipase Lip182 had similar elasticity as the control and the combination of JPO172 and Lip182 was similar to JPO172 alone.

TABLE 72
Firmness (g) on day 1 and 7 of bread with
enzyme treatments according Table 71.

| Treatment | Day 1 | Day 7 |
|---|---|---|
| Control | 291 | 936 |
| Opticake 50 BG, 200 MANU/kg flour | 170 | 333 |
| Lip182, 2.5 PLA(B)/kg flour | 340 | 875 |
| Lip182, 5 PLA(B)/kg flour | 326 | 1031 |
| JPO172, 25 mgEP/kg flour | 198 | 481 |
| JPO172, 50 mgEP/kg flour | 172 | 495 |
| JPO172, 25 mgEP/kg flour − Lip182, 2.5 PLA(B)/ | 217 | 728 |
| kg flour | | |
| JPO172 25 mgEP/kg flour − Lip182, 5 PLA(B)/ | 193 | 578 |
| kg flour | | |
| JPO172, 50 mgEP/kg flour − Lip182, 2.5 PLA(B)/ | 171 | 565 |
| kg flour | | |
| JPO172, 50 mgEP/kg flour − Lip182, 5 PLA(B)/ | 158 | 377 |
| kg flour | | |

TABLE 73
Elasticity (%) on day 1 and 7 of bread with
enzyme treatments according Table 71.

| Treatment | Day 1 | Day 7 |
|---|---|---|
| Control | 58.2 | 45.8 |
| Opticake 50 BG, 200 MANU/kg flour | 60.8 | 55.8 |
| Lip182, 2.5 PLA(B)/kg flour | 57.6 | 46.4 |
| Lip182, 5 PLA(B)/kg flour | 57.2 | 46.7 |
| JPO172, 25 mgEP/kg flour | 63.4 | 51.3 |
| JPO172, 50 mgEP/kg flour | 66.0 | 57.7 |
| JPO172, 25 mgEP/kg flour − Lip182, 2.5 PLA(B)/ | 61.7 | 49.9 |
| kg flour | | |
| JPO172 25 mgEP/kg flour − Lip182, 5 PLA(B)/ | 61.9 | 51.0 |
| kg flour | | |
| JPO172, 50 mgEP/kg flour − Lip182, 2.5 PLA(B)/ | 64.8 | 55.2 |
| kg flour | | |
| JPO172, 50 mgEP/kg flour − Lip182, 5 PLA(B)/ | 64.7 | 57.6 |
| kg flour | | |

### Example 20. Freshness Effect of AMG Combined with Gluzyme Fortis

Bread was baked in a straight dough mini baking process with a recipe according to

Table 74. The bread was baked in lidded tins in order to have the same volume of all bread. The ingredients were mixed in a spiral mixer into a dough for 4 minutes at 90 rpm. The doughs were divided into 20 g pieces, rounded and place in baking tins. The tins with the doughs were proofed on a conveyor belt for 55 min at 36° C. and 80% relative humidity. The proofed doughs were baked in mini tunnel oven for 12 min at 210° C.

TABLE 74

| | Bakers % |
|---|---|
| Flour (Kolibri, Meneba, NL) | 100 |
| Water | 58 |
| Yeast | 4.5 |
| Sucrose | 1.5 |
| Salt | 1.5 |
| Ascorbic acid | 0.04 |
| Calcium Propionate | 0.3 |
| Fungamyl ® 4000SG | 8 ppm |
| Panzea ® BG | 25 ppm |

TABLE 75
Various treatments were prepared with different enzymatic additions.
Additional water was added to achieve similar dough rheology.

| | Opticake 50BG, | JPO172, | Gluzyme Fortis, | |
|---|---|---|---|---|
| | MANU/kg | mgEP/kg | GODU/kg of | Additional |
| Treatment | flour | flour | flour | water, % |
| 1 | | | | |
| 2 | 200 | | | |
| 3 | | | 150 | 0.5 |
| 4 | | | 300 | 1.5 |
| 5 | | 25 | | |
| 6 | | 50 | | |
| 7 | | 25 | 150 | 0.5 |
| 8 | | 25 | 300 | 1.5 |
| 9 | | 50 | 150 | 0.5 |
| 10 | | 50 | 300 | 1.5 |

The doughs were baked and the resulting breads were packed 0.5 hours after baking in sealed plastic bags and stored at room temperature until analysis.

The texture of each bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product.

A standard method for measuring firmness and elasticity is based on force-deformation of the baked product. A force-deformation of the baked products may be performed with a 20 mm diameter spherical probe. The force on the probe is recorded as it is pressed down 40% strain on a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 40% strain (corresponding to force at time=40 s for a bread slice of 25 mm thickness) divided by the force needed to press the probe 10 mm into the crumb (corresponding to force at time=10 s for a bread slice of 25 mm thickness) times 100.

The results from the texture analysis can be found in Table 76 (firmness) and Table 77 (elasticity).

Fresh bread without enzyme (control) has low firmness and high elasticity, as the bread is stored the firmness increase over time and the elasticity decrease. Traditional AMGs used in baking applications (for example Goldcrust) does not impact the firmness or elasticity (Example 7).

The AMG JPO172 improved (reduced) initial firmness and the increase in firmness over time. The glucose oxidase (Gluzyme Fortis) alone reduced firmness compared to a control bread to some degree. The combination of glucose oxidase and JPO172 resulted in the bread with lowest firmness on both day 1 and 7.

The AMGs JPO172 improved (increased) the initial elasticity and prevented the loss of elasticity over time. The glucose oxidase (Gluzyme Fortis) alone had similar elasticity as the control and the combination of JPO172 and the glucose oxidase was similar to JPO172 alone.

TABLE 76
Firmness (g) on day 1 and 7 of bread with
enzyme treatments according Table 75.

| | Day 1 | Day 7 |
|---|---|---|
| Blank | 281 | 944 |
| Opticake ® 50 BG, 200 MANU/kg flour | 169 | 423 |
| Gluzyme Fortis ®, 150 GODU/kg flour | 216 | 825 |
| Gluzyme Fortis ®, 300 GODU/kg flour | 239 | 851 |
| JPO172, 25 mgEP/kg flour | 157 | 521 |
| JPO172, 50 mgEP/kg flour | 154 | 390 |
| Gluzyme Fortis ®, 150 GODU/kg flour + | 189 | 572 |
| JPO172, 25 mgEP/kg flour | | |
| Gluzyme Fortis ®, 300 GODU/kg flour + | 159 | 456 |
| JPO172, 25 mgEP/kg flour | | |
| Gluzyme Fortis ®, 150 GODU/kg flour + | 155 | 445 |
| JPO172, 50 mgEP/kg flour | | |
| Gluzyme Fortis ®, 300 GODU/kg flour + | 128 | 354 |
| JPO172, 50 mgEP/kg flour | | |

TABLE 77
Elasticity (%) on day 1 and 7 of bread with
enzyme treatments according Table 75.

| | Day 1 | Day 7 |
|---|---|---|
| Blank | 55.8 | 44.1 |
| Opticake ® 50 BG, 200 MANU/kg flour | 57.8 | 52.0 |
| Gluzyme Fortis ®, 150 GODU/kg flour | 57.1 | 44.3 |
| Gluzyme Fortis ®, 300 GODU/kg flour | 57.4 | 44.1 |
| JPO172, 25 mgEP/kg flour | 61.8 | 52.1 |
| JPO172, 50 mgEP/kg flour | 64.4 | 57.0 |
| Gluzyme Fortis ®, 150 GODU/kg flour + | 60.8 | 48.4 |
| JPO172, 25 mgEP/kg flour | | |
| Gluzyme Fortis ®, 300 GODU/kg flour + | 61.9 | 49.5 |
| JPO172, 25 mgEP/kg flour | | |
| Gluzyme Fortis ®, 150 GODU/kg flour + | 64.7 | 56.0 |
| JPO172, 50 mgEP/kg flour | | |
| Gluzyme Fortis ®, 300 GODU/kg flour + | 63.4 | 55.4 |
| JPO172, 50 mgEP/kg flour | | |

### Example 21. Sensory Comparison of Freshness Effect of AMG Goldcrust®, AMG NL, AMG AnPAV498 and JPO124 in Sponge and Dough Recipe

Bread was baked in a sponge and dough process with a recipe according to table 78. The bread was baked in lidded tins in order to have the same volume of all bread. The ingredients of the sponge were mixed in a pin mixer into a dough for 2+1 min at 50 respectively 150 rpm. The sponge was proofed for 2 hours at 27° C. and 75% rH. The sponge was placed in the pin mixer with the rest of the ingredients of the dough and mixed into a dough for 1+3 minutes at 50 and 150 rpm respectively.

The doughs were divided into 400 gram pieces, rounded, sheeted and place in baking tins with lid. The tins with the doughs were proofed for 60 min at 43° C. and 80% relative humidity. The proofed doughs were baked in a revolving oven for 20 min at 215° C.

TABLE 78
Recipe

| | |
|---|---|
| Ingredient | Amount |

Sponge

| | |
|---|---|
| Flour, % | 70 |
| Water, % | 40.6 |
| Dry yeast, % | 2.0 |
| SSL, % | 0.5 |
| Soybean oil, % | 3 |
| Calcium propionate, % | 0.1 |

Dough

| | |
|---|---|
| Flour, % | 30 |
| Water, % | 16.4 |
| Sugar, % | 8 |
| Salt, % | 2.0 |
| Calcium Propionate, % | 0.4 |
| Ascorbic acid, ppm | 60 |
| Fungamyl ® 4000 SG, ppm | 7 |
| Panzea ® BG, ppm | 25 |

TABLE 79
Treatments were prepared with different enzymatic additions

| | | | | | |
|---|---|---|---|---|---|
| Dough | 1 | 2 | 3 | 4 | 5 |

| | | | | |
|---|---|---|---|---|
| Control (no | | | | |
| glucoamylase) | | | | |
| AMG | 50 | | | |
| Goldcrust ®, | | | | |
| mgEP/kg | | | | |
| AMG NL mg EP/kg | | 50 | | |
| AMG | | | 50 | |
| AnPAV498 mgEP/kg | | | | |
| JPO124 mgEP/kg | | | | 50 |

### Sensory Evaluation Method

Sensory evaluation was performed on day 1 and day 7. A training session was held prior to evaluation, identifying the relevant attributes and procedures (Table 80). Texture was evaluated by hand. 5 trained assessors participated in the evaluation. Each assessor was served 2 slices of each bread type. Samples were served blind, 3-digit coded, and in random order. Intensity of the sensory attributes were evaluated on a 1-9 point intensity scale ranging from little to very intense. Two sensory replicates were performed on each evaluation day.

TABLE 80
Description of sensory attributes, procedures and evaluation

| Attribute | Procedure | Evaluate |
|---|---|---|
| Moist | Touch the surface of the | Degree of moistness/cooling |
| | bread, hold for 3 sec | perceived |
| Soft | Gently compress the bread | Ease of compressing the |
| | | crumb |
| Resilient/ | Compress the slice | Degree to which the crumb |
| springy | completely | recovers to original shape |

Use the other bread slice

| | | |
|---|---|---|
| Foldable | Fold the bread slice | Extend of coherency at the fold |

### Sensory Results

JPO124 scored the highest on Moist, Soft and Foldable day 7, followed by AMG AnPAV498.

TABLE 81
Mean values of sensory scores of the bread day 1.

| Day 1 | Moist | Soft | Resilient/springy | Foldable |
|---|---|---|---|---|
| Control | 6.7 | 6.6 | 5.0 | 5.3 |
| 50 mgEP/kg Goldcrust ® | 6.8 | 6.8 | 5.1 | 6.3 |
| 50 mgEP/kg JPO124 | 7.4 | 7.1 | 5.3 | 6.5 |
| 50 mgEP/kg AMG NL | 7.0 | 6.9 | 4.4 | 5.7 |
| 50 mgEP/kg AMG | 6.8 | 6.8 | 5.4 | 5.9 |
| AnPAV498 | | | | |

TABLE 82
Mean values of sensory scores of the bread day 7.

| Day 7 | Moist | Soft | Resilient/springy | Foldable |
|---|---|---|---|---|
| Control | 3.2 | 3.5 | 4.8 | 1.2 |
| 50 mgEP/kg Goldcrust ® | 3.7 | 3.9 | 5.3 | 1.3 |
| 50 mgEP/kg JPO124 | 4.6 | 5.2 | 4.5 | 2.7 |
| 50 mgEP/kg AMG NL | 3.7 | 3.6 | 4.5 | 1.6 |
| 50 mgEP/kg AMG | 4.0 | 4.3 | 4.3 | 2.3 |
| AnPAV498 | | | | |

### Example 22. Freshness Effect of JPO172 in Low pH Mixed Rye/Wheat Sour Dough Bread

Bread was baked in a straight dough process with a recipe according to Table 83. Nine different treatments were done according to table 84. The ingredients were mixed in a spiral mixer into a dough for 6+4 min at 17 respectively 35 rpm. The doughs were divided into 650 g pieces, rounded, sheeted and place in baking tins. The pH of the final dough was of 4.3. The bread was baked in lidded tins in order to have the same volume of all bread. The tins with the doughs were proofed for 60 min at 32° C. and 85% relative humidity. The proofed doughs were baked in a deck oven for 20 min at 225° C. 10

TABLE 83
Recipe

| Ingredients | Amount, % |
|---|---|
| Flour 1 (Rye flour) | 50 |
| Flour 2 (Wheat flour) | 50 |
| Water | 65.5 |
| Yeast 70% water | 3 |
| Rye sour | 2.24 |
| Salt | 2 |
| Ascorbic acid | 0.06 |
| Calcium Propionate | 0.25 |
| Vital Wheat Gluten | 1 |
| Vinegar | 1 |
| Citric acid | 0.3 |

TABLE 84
Treatments.

| | Pentopan | JPO172 mg | |
|---|---|---|---|
| | 500, ppm | EP/kg | |
| | (flour) | flour | Sample name |
| 1 | 50 | | Control |
| 2 | 50 | 12.5 | 12.5 mgEP/kg flour JPO172 |
| 3 | 50 | 25 | 25 mgEP/kg flour JPO172 |
| 4 | 50 | 50 | 50 mgEP/kg flour JPO172 |
| 5 | 50 | 100 | 100 mgEP/kg flour JPO172 |

After baking, the bread was allowed to cool down for 2 hours and placed in sealed plastic bags. The bread was stored at room temperature until analysis.

The texture of each bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product. A standard method for measuring firmness and elasticity is based on force-deformation of the baked product. A force-deformation of the baked products may be performed with a 40 mm diameter cylindrical probe. The force on the cylindrical probe is recorded as it is pressed down at a deformation speed of 1 mm/second. The probe is then kept in this position for 30 seconds while the force is recorded and then the probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 40% strain (corresponding to force at time=40 s for a bread slice of 25 mm thickness) divided by the force needed to press the probe 10 mm into the crumb (corresponding to force at time=10 s for a bread slice of 25 mm thickness) times 100.

The results from the texture analysis can be found in Table 85 (firmness) and Table 86 (elasticity).

Fresh bread without any treatment (Control) have a low firmness and high elasticity, as the bread is stored the bread becomes more firm and loses elasticity. Bread with JPO172 was less firm and had a higher elasticity after baking. The change in firmness and elasticity over time was also reduced compared to a control bread, making the bread with JPO172 less firm and more elastic on day 7 compared to a control bread on day 1.

TABLE 85
Effect on various treatments on Firmness

| Treatment | Day 1 | Day 3 | Day 7 |
|---|---|---|---|
| Control | 1316 | 2331 | 3040 |
| 12.5 mgEP/kg flour JPO172 | 1012 | 1592 | 2072 |
| 25 mgEP/kg flour JPO172 | 872 | 1217 | 1668 |
| 50 mgEP/kg flour JPO172 | 858 | 1129 | 1312 |
| 100 mgEP/kg flour JPO172 | 879 | 992 | 1097 |

TABLE 86
Effect on various treatments on Elasticity.

| Treatment | Day 1 | Day 3 | Day 7 |
|---|---|---|---|
| Control | 55.4 | 44.9 | 43.6 |
| 12.5 mgEP/kg flour JPO172 | 61.9 | 54.0 | 48.2 |
| 25 mgEP/kg flour JPO172 | 64.6 | 60.6 | 54.6 |
| 50 mgEP/kg flour JPO172 | 65.4 | 63.1 | 61.1 |
| 100 mgEP/kg flour JPO172 | 66.2 | 65.6 | 65.0 |

### Example 23. Freshness Effect of JPO172 in Tortillas

Tortillas were made using the recipe in Table 87, different enzymatic solutions were added according to table 88. The ingredients were mixed in a pin mixer for 1+6 minutes at low and high speed respectively. The doughs were allowed to rest for 2 minutes. The dough was divided into 30 g pieces and shaped into rolls. The tortillas were baked in a two-step process where the dough pieces first went through a hot press at 160° C. for 6 seconds, secondly the tortilla was baked for 20 seconds and flipped over and baked for another 20 seconds.

TABLE 87
Recipe.

| Ingredient | Amount, % |
|---|---|
| Flour | 100.0 |
| Water | 54 |
| Baking powder | 3.0 |
| Glycerol | 4.5 |
| Salt | 2.0 |
| Sugar | 2.00 |
| Citric acid | 0.40 |
| Calcium propionate | 0.50 |
| DMG | 0.50 |
| SSL | 0.25 |
| Guar Gum | 0.30 |
| Sunflower oil | 6.00 |

TABLE 88
Various treatments were prepared with
different enzymatic additions.

| | JPO172 | Sensea ® | |
|---|---|---|---|
| Enzyme | mg EP/ | wrap | |
| Dough# | kg flour | ppm (flour) | Sample name |
| 1 | | | control |
| 2 | 400 | | 400 mgEP/kg JPO172 |
| 3 | 2000 | | 2000 mgEP/kg JPO172 |
| 4 | | 200 | 200 ppm Sensea Wrap |
| 5 | | 400 | 400 ppm Sensea Wrap |

The tortillas were allowed to cool down for 30 minutes after baking and the placed in a sealed plastic bag that were stored at room temperature until analysis.

The texture properties of tortillas were evaluated with a texture analyzer (Stable Microsystems, Godalming, UK) using the Tortilla/Pastry Burst Rig (HDP/TPB). In the test procedure, the sample is held between two plates and the 1″ spherical probe is driven through the center. The force and distance to extend the sample are measured and used as an indication of ‘deformation resistance’ and ‘extensibility’, respectively.

The tortillas are typically used as wraps where the tortilla is wrapped around different types of fillings. An important parameter is the extensibility which describes the resistance to rupture. A fresh tortilla is extensible. However, it loses this extensibility quite rapidly upon storage, as can be seen in table 90. The addition of JPO172 results in a tortilla that has an extensibility similar to a freshly baked tortilla after 28 days.

TABLE 89
Deformation resistance, g of tortilla

| Treatment | Dose | Day 1 | Day 14 | Day 28 |
|---|---|---|---|---|
| JPO172, | 0 | 663 | 482 | 333 |
| mgEP/kg flour | 400 | 727 | 634 | 492 |
| Sensea Wrap, | 2000 | 868 | 616 | 498 |
| ppm | 0 | 663 | 482 | 333 |
| | 200 | 649 | 543 | 420 |
| | 400 | 689 | 542 | 445 |

TABLE 90
Extensibility in mm of tortilla

| Treatment | Dose | Day 1 | Day 14 | Day 28 |
|---|---|---|---|---|
| JPO172, | 0 | 19 | 7 | 5 |
| mgEP/kg flour | 400 | 16 | 12 | 9 |
| | 2000 | 19 | 15 | 14 |
| Sensea ® | 0 | 19 | 7 | 5 |
| Wrap, ppm | 200 | 18 | 13 | 9 |
| | 400 | 21 | 14 | 13 |

### Example 24. Freshness Effect of JPO172 in Brioche

Bread was baked in a straight dough process with a recipe according to Table 91. Eight different treatments were done according to table 92. The ingredients were mixed in a spiral mixer into a dough for 4+8 min at 17 respectively 35 rpm. The doughs were divided into 420 g pieces, rounded, sheeted and place in baking tins. The doughs were proofed for 2.5 hours at 30° C. and 75% rH. The bread was baked for 34 minutes at 175° C.

TABLE 91
Recipe.

| Recipe: | % |
|---|---|
| Water | 33 |
| Flour | 97 |
| Vital gluten | 3 |
| Salt (0.3% less due to salted butter) | 2 |
| Whole Egg (without preservative) | 20 |
| Dried yeast (sugar tolerant - | 2 |
| GOLD) | |
| Sugar | 25 |
| Butter (Salted Lurpak ®) | 20 |
| Ascorbic-Acid (ppm) | 0.006 |

TABLE 92
Treatments

| | Fungamyl | Panzea BG, | |
|---|---|---|---|
| | 4000 SG, | ppm | JPO172 mg |
| | ppm (flour) | (flour) | EP/kg flour |
| 1 | 6 | 20 | |
| 2 | 6 | 20 | 50 |
| 3 | 6 | 20 | 75 |
| 4 | 6 | 20 | 100 |
| 5 | 6 | 20 | 150 |
| 6 | 6 | 20 | 200 |

After baking the bread was allowed to cool down for 2 hours and placed in sealed plastic bags. The bread was stored at room temperature until analysis.

The texture of each bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product. A standard method for measuring firmness and elasticity is based on force-deformation of the baked product. A force-deformation of the baked products may be performed with a 34 mm diameter cylindrical probe. The force on the cylindrical probe is recorded as it is pressed down 28% strain a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 28% strain (corresponding to force at time=40 s for a bread slice of 25 mm thickness) divided by the force needed to press the probe 10 mm into the crumb (corresponding to force at time=10 s for a bread slice of 25 mm thickness) times 100.

The results from the texture analysis can be found in Table 93 (firmness) and Table 94 (elasticity).

Fresh bread without any treatment (Control) have a low firmness and high elasticity after baking, as the bread is stored the bread becomes more firm and loses elasticity. Bread with JPO172 was less firm and had a higher elasticity after baking compared to the control. As the bread with JPO172 was stored the firmness and elasticity changed only slightly, resulting in a Brioche on day 60 with JPO172 having similar Firmness and better Elasticity as a control on day 1.

TABLE 93
Effect on various treatments on Firmness.

| Treatment | Day 1 | Day 21 | Day 39 | Day 60 |
|---|---|---|---|---|
| Control | 244 | 918 | 1111 | 1151 |
| 50 mgEP JP0172\Kg flour | 213 | 407 | 502 | 566 |
| 75 mgEP JP0172\Kg flour | 157 | 324 | 307 | 414 |
| 100 mgEP JP0172\Kg flour | 126 | 306 | 269 | 348 |
| 150 mgEP JP0172\Kg flour | 144 | 300 | 316 | 299 |
| 200 mgEP JP0172\Kg flour | 159 | 321 | 317 | 334 |

TABLE 94
Effect on various treatments on Elasticity.

| Treatment | Day 1 | Day 21 | Day 39 | Day 60 |
|---|---|---|---|---|
| Control | 52.3 | 42.2 | 40.4 | 40.9 |
| 50 mgEP JP0172\Kg flour | 58.0 | 51.9 | 51.0 | 50.0 |
| 75 mgEP JP0172\Kg flour | 59.9 | 55.5 | 54.9 | 53.1 |
| 100 mgEP JP0172\Kg flour | 60.0 | 56.7 | 55.5 | 54.8 |
| 150 mgEP JP0172\Kg flour | 60.3 | 57.2 | 56.5 | 55.9 |
| 200 mgEP JP0172\Kg flour | 60.5 | 56.9 | 56.6 | 55.9 |

### Example 25. JPO124 and JPO172 in Lebanese Double Layer Flat Bread

Lebanese double layer flat bread was baked in a straight dough process with ingredients according to table 95. Seven different treatments were done according to table 96. The ingredients were mixed in a spiral mixer into a dough for 2.5 minutes at 35 rpm. The dough were proofed for 40 minutes at 32° C. and 82% rH. The dough was rolled out rolled out to a thickness of 2 mm, and a 20 cm circular dough piece was cut out from the sheet. The circular dough pieces were proofed at room temperature for 20 minutes. The dough was placed in an oven at 750° C. and baked for 9 seconds.

TABLE 95
Recipe.

| Ingredient | Amount, % |
|---|---|
| Flour | 100.0 |
| Water | 51 |
| Dry instant Yeast | 0.7 |
| Sucrose | 4.0 |
| Salt | 0.4 |
| Calcium propionate | 0.20 |

TABLE 96
Treatments.

| | JPO 124, | JPO 172, | |
|---|---|---|---|
| | mg EP/ | mg EP/ | |
| | kg flour | kg flour | Sample name |
| 1 | | | control |
| 2 | | 400 | 400 mgEP/kg JPO172 |
| 3 | | 2000 | 2000 mgEP/kg JPO172 |
| 4 | | 4000 | 4000 mgEP/kg JPO172 |
| 5 | 400 | | 400 mgEP/kg JPO124 |
| 6 | 4000 | | 4000 mgEP/kg JPO124 |

The flat breads were allowed to cool down for 30 minutes after baking and then placed in a sealed plastic bag that was stored at room temperature until analysis.

The texture properties of Lebanese flat bread were evaluated with a texture analyzer (Stable Microsystems, Godalming, UK) on day 3 using the Tortilla/Pastry Burst Rig (HDP/TPB). In the test procedure, the sample is held between two plates and the 4 mm spherical probe is driven through the center. The force and distance to extend the sample are measured and used as an indication of ‘deformation resistance’ and ‘extensibility’, respectively.

Sensory evaluation was performed on day 3. A training session was held prior to evaluation, identifying the relevant attributes and procedures (Table ZZ). Texture was evaluated by hand. 4-5 trained assessors participated in the evaluation. Each assessor was served 2 slices without crust of each bread type. Samples were served blind, 3-digit coded, and in random order. The intensities of the sensory attributes were evaluated on a 1-9 point intensity scale ranging from little to very intense. Two sensory replicates were performed on each evaluation day.

TABLE 97
Sensory evaluations.

| Attribute | Procedure | Evaluate |
|---|---|---|
| Soft | Gently compress the bread | Ease of compressing the |
| | | flat bread |
| Flexible | Place the flat bread on the | How much the flat bread |
| | tip of your fingers | bends down |
| Foldable | Roll the flat bread around | Degree of cracking |
| | a 20 mm round stick | |
| Elastic | Grab the flat bread at the edges | Resistance to extension |
| | and stretch the flat bread | |
| Layer | Open the flat bread at an edge | Ease of layer separation |
| separation | and separate the layers of | |
| | the flat bread | |

The results for the sensory evaluation can be found in table 98 and the results from the texture evaluation can be found in table 99. The bread without any enzymes added (control) was scored low (2-3) on all sensory parameters. The flat bread with JPO172 and JPO124 scored higher on all parameters, the higher the dosage the higher the score. The improvement detected in the sensory evaluation was also seen in the texture analysis, where the bread with JPO124 or JPO172 had higher extensibility compared to flat bread without any enzyme (Control).

TABLE 98
Sensory evaluation of Lebanese flat bread on day 3.

| | | Flex- | | | Layer |
|---|---|---|---|---|---|
| | Soft | ible | Foldable | Elastic | separation |
| Control | 2.8 | 2.6 | 2.0 | 2.2 | 2.6 |
| 400 mgEP/kg JPO172 | 3.3 | 2.4 | 3.5 | 2.9 | 4.9 |
| 2000 mgEP/kg JPO172 | 6.6 | 4.2 | 6.9 | 5.4 | 6.8 |
| 4000 mgEP/kg JPO172 | 6.6 | 4.0 | 6.9 | 4.9 | 6.7 |
| 400 mgEP/kg JPO124 | 4.5 | 3.7 | 3.5 | 3.3 | 5.9 |
| 4000 mgEP/kg JPO124 | 7.1 | 5.5 | 7.1 | 5.3 | 6.9 |

TABLE 99
Texture evaluation of Lebanese flat bread on day 3.

| | Toughness | Extensibility |
|---|---|---|
| Control | 271 | 3.8 |
| 400 mgEP/kg JPO172 | 266 | 5.1 |
| 2000 mgEP/kg JPO172 | 204 | 5.0 |
| 4000 mgEP/kg JPO172 | 217 | 5.5 |
| 400 mgEP/kg JPO124 | 323 | 4.9 |
| 4000 mgEP/kg JPO124 | 268 | 6.4 |

1. A method of producing a baked or par-baked product, said method comprising:
a) providing a dough comprising a mature thermostable variant of a parent glucoamylase at least 70% identical to SEQ ID NO:1, SEQ ID NO:6, SEQ ID NO:7 or SEQ ID NO:8; and
b) baking or par-baking the dough to produce a baked or par-baked product.

2. The method according to claim 1, wherein the baked or par-baked product is a type of bread, preferably a pan bread, toast bread, open bread, buns, Fino bread, Hammam bread, Samoli bread, baguettes, brioche, hamburger buns, rolls, brown bread, whole meal bread, rich bread, bran bread, flat bread, tortilla or biscuit, cake, or a patisserie.

3. The method according to claim 1, wherein the parent glucoamylase is from a species of Penicillium, preferably from Penicillium oxicalum, Penicillium miczynskii, Penicillium russellii or Penicillium glabrum.

4. The method according to claim 1, wherein the mature variant comprises at least one amino acid modification in one or more or all of the positions corresponding to positions 1, 2, 4, 6, 7, 11, 31, 34, 65, 79, 103, 132, 327, 445, 447, 481, 566, 568, 594 and 595 in SEQ ID NO:1.

5. The method according to claim 4, wherein the at least one amino acid modification comprises a substitution in one or more or all of the positions corresponding to positions 1, 2, 4, 11, 65, 79 and 327 in SEQ ID NO:1.

6. The method according to claim 4, wherein the at least one amino acid modification comprises a substitution in one or more or all of the positions corresponding to positions 1, 6, 7, 31, 34, 79, 103, 132, 445, 447, 481, 566, 568, 594 and 595 in SEQ ID NO:1.

7. The method according to claim 1, wherein the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1.

8. The method according to claim 1, wherein the mature thermostable variant has a thermostability improvement (Td) over its parent of at least 3° C.

9. The method according to claim 1, wherein the mature thermostable variant has a relative activity at 91° C. of at least 150 compared to its parent.

10. The method according to claim 1, wherein the baked or par-baked product after final bake-off has a reduced initial firmness and/or an increased initial elasticity, and/or a reduced increase in firmness and/or a higher elasticity after 1, 7 or 14 days, when cooled to room temperature, packed in a sealed container and stored at room temperature until analysis, compared to a control made without any added glucoamylase.

11. The method according to claim 1, wherein the baked or par-baked product after final bake-off has at least the same sweetness as a control product made with double the amount of the mature glucoamylase the amino acid sequence of which is shown in SEQ ID NO:10.

12. The method according to claim 1, wherein the mature thermostable variant glucoamylase enzyme is comprised in the dough in an amount of 0.01-1,000 mg enzyme protein (mgEP) per kg flour.

13. The method according to claim 1, wherein the dough also comprises one or more additional enzyme selected from the group consisting of a alpha-amylase, maltogenic amylase, raw-starch degrading alpha-amylase, beta amylase, aminopeptidase, carboxypeptidase, catalase, chitinase, cellulytic enzyme, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase; wherein the one or more additional enzyme is comprised in an amount of 0.01-1,000 mg enzyme protein (mgEP) per kg flour.

14. A baking composition comprising a mature thermostable variant of a parent glucoamylase as defined in claim 1.

15. The baking composition of claim 14, which also comprises one or more additional enzyme selected from the group consisting of a alpha-amylase, maltogenic amylase, beta amylase, aminopeptidase, carboxypeptidase, catalase, cellulytic enzyme, chitinase, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase.

16. The baking composition of claim 14, which also comprises flour, sugar, yeast, salt and/or fat.

17. (canceled)

18. (canceled)

19. The method according to claim 4, wherein the at least one amino acid modification comprises a substitution in one or more or all of the positions corresponding to R1A, P2N, P4S, P11F, T65A, K79V and Q327F in SEQ ID NO:1.

20. The method according to claim 4, wherein the at least one amino acid modification comprises a substitution in one or more or all of the positions corresponding to R1A, G6S, G7T, R31F, K34Y, K79V, S103N, A132P, D445N, V447S, S481P, D566T, T568V, Q594R and F595S in SEQ ID NO:1.

21. The method according to claim 4, wherein the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1.


SEQUENCE LISTING (10 sequences)

SEQ ID NO: 1 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 2 - protein, 595 aa, Artificial sequence
  AMG NL
RPDPKGGNLTPFIHKEGERSLQGILDNLGGRGKKTPGTAAGLFIASPNTENPNYYYTWTRDSALTAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 3 - protein, 595 aa, Artificial sequence
  AMG anPAV498
RNDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 4 - protein, 595 aa, Artificial sequence
  AMG JPO-001
ANDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 5 - protein, 595 aa, Artificial sequence
  AMG JPO-124
ANDSKSTNLTFFIHKEGERSLQGILDNLGGFGKYTPGTAAGLFIASPNTENPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKTKCVPAKDIVPITFYLIENTYYGENVFMSGNITALGNWDAKKGFPLTANLYTQDQNLWFASVEFIPAGTPFEYKYYKVEPNGTIVWEKGPNRVFVAPTGCPVQPHSNDVWRS

SEQ ID NO: 6 - protein, 595 aa, Artificial sequence
  AMG JPO-172
ANDSKSTNLTFFIHKEGERSLQGILDNLGGFGKYTPGTAAGLFIASPNTRNPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKPKCVPAKDIVPITFYLIANTYYGENVFMSGNITALGNWDAKKGFPLTANLYTQDQPLWFASVEFIPAGTPFEYKYYKVEPNGTIVWEKGPNRVFVAPTGCPVQPHSNDVWRS

SEQ ID NO: 7 - protein, 593 aa, Penicillium miczynskii
NPTSKDAKMASYISKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLISHGSKSEAANIMWPIIANDLAYVGQYWNNTGFDLWEEVDGSSFFTLAVQHRSLVQGASLAKKLGKSCPACVSQAPQILCFLQSFWNGNYITANINLDTSRSGIDLNSILGSIHTFDPEASCDDSTFQPCSARALANHKVYVDAFRSIYKVNAGLSNGSAANVGRYPEDVYQGGNPWYLATLASAELLYDALYQWSKIGKLDVTKTSLAFFKDFDMAVKTGTYSAHSSTYKSLTSAIRTYADDFIGLVQDYTPSNGSLAEQYDRNTGIPLSANDLTWSYASFITAIQRRASVVPASWGEKSANVVPTICSASPVTGTYQAAPSVFPTTTGCVPATSIVPITFYLTETTFYGENVYMTGNISALGNWDTNNGFPLTANLYTDSDHLWFASVELVPAGTPFEYKYYKVEPNGTVIWENGENRVYVAPTGCPIQPSQTDVWR

SEQ ID NO: 8 - protein, 590 aa, Penicillium russellii
SKDGNLASYIAKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLVSHGQKSEATNIMWPIIANDLAYVGQYWNKTGFDLWEEVDGSSFYTLAVQHRALVQGASLAKKLGKSCTACVSQAPQILCFLQSFWNGNYITANINLDTSRSGIDLNSILGSIHTFDPEASCDDSTFQPCSARALANHKVYVDAFRSIYGVNAGLSNGTAANVGRYPEDVYQGGNPWYLATLAAAELLYDALYQWNQIGKLDVTKTSLAFFKDFDAAVKTGTYSAHSSAYRTLTSAIRTYADDFISLVQHYTPSNGSLAEQYDRDTGIPLSANDLTWSYASFITAIERRASVVPASWGEKSANVVPTTCSASPVTGTYVAATSVFPTTTGCVPATSIVPITFYLTESTFYGENVYMTGNISALGNWDTSSGFPLTANLYTDSDHLWFASVELVPAGTPFEYKYYKVEPNGTVIWENGENRVYVAPTGCPIQPSQTDIWR

SEQ ID NO: 9 - protein, 594 aa, Penicillium glabrum
NPFNSLDSFILKEGARSYQGIIDNLGNKGVKAPGTAAGLFVASPNTANPDYFYTWTRDSALTFKCLIDLFDGGSTEFGLKNSELETDIRNYVSSQAVLQNVSNPSGTLEDGTGLGEPKFEVDLNPFTGSWGRPQRDGPALRATALITYTNYLLSQGQKSEAVNIMWPIISNDLAYVGQYWNDTGFDLWEETDGSSFFTLAVQHRALVQGATLAQKLGKSCAACSSQAPQILCFLQSFWNGKYITANINLDTSRTGIDANTLLGSIHTFDPEAACDDSTFQPCSARALANHKVYVDAFRSIYKINSGIAEGSPANVGRYPEDVYQGGNPWYLTTLASAELLYDALYQWNKIGGLDVTETSLAFFKDFHSSVKTGSYSAHSQTYKTLTSAIRTYADGFVGLVQKYTPANGSLAEQYNRNTSVPLSANDLTWSFASFLTAIQRRESIVPGSWGEKSANTVPTTCSASPVTGTYKAATSTFPTSTAGCVPATDVVPITFYLIENTYYGENVYMTGNISALGNWDTSDGLALDAGLYTETDNLWFGTLELVTAGTPFEYKYYKIEPNGTVTIWESGDNRVSVVPTGCPIQPSLHDVWRS

SEQ ID NO: 10 - protein, 591 aa, Rasamsonia emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ